(19) United States
(12) Patent Application Publication
(10) Pub. No.: US 2020/0109452 A1
(43) Pub. Date: Apr. 9, 2020

(54) METHOD OF DETECTING A FETAL CHROMOSOMAL ABNORMALITY

(71) Applicant: Premaitha Limited, Manchester (GB)

(72) Inventors: Francesco CREA, Manchester (GB); Matthew FORMAN, Manchester (GB); Michael RISLEY, Manchester (GB); Rachel SHELMERDINE, Manchester (GB)

(21) Appl. No.: 16/499,849

(22) PCT Filed: Mar. 29, 2018
(86) PCT No.: PCT/GB2018/050855

§ 371 Date: Sep. 30, 2019

(30) Foreign Application Priority Data
Mar. 31, 2017 (GB) 1705281.2
Nov. 10, 2017 (GB) 1718623.0

(51) Int. Cl. C12Q 1/6883; G16B 30/10; G16B 20/20; C12N 15/10; C12Q 1/6879

(52) U.S. Cl. CPC C12Q 1/6883; G16B 30/10; C12Q 2600/156; C12N 15/1034; C12Q 1/6879; G16B 20/20

(57) ABSTRACT

The invention relates to a novel method of detecting a fetal chromosomal abnormality, in particular, the invention relates to the detection of trisomy 21 (Down's syndrome) which comprises enrichment of the analysed fragment sizes from approximately 100 bp to approximately 150 bp. The invention also relates to kits for performing said method. The invention also relates to a method of predicting the gender of a fetus within a pregnant female subject.

### FIELD OF THE INVENTION

The invention relates to a novel method of detecting a fetal chromosomal abnormality, in particular, the invention relates to the detection of trisomy 21 (Down's syndrome) which comprises enrichment of the analysed fragment sizes from approximately 100 bp to approximately 150 bp. The invention also relates to kits for performing said method. The invention also relates to a method of predicting the gender of a fetus within a pregnant female subject.

### BACKGROUND OF THE INVENTION

Down's Syndrome is a relatively common genetic disorder, affecting about 1 in 800 live births. This syndrome is caused by the presence of an extra whole chromosome 21 (trisomy 21, T21), or less commonly, an extra substantial portion of that chromosome. Trisomies involving other autosomes (i.e. T13 or T18) also occur in live births, but more rarely than T21.

Generally, conditions where there is fetal aneuploidy resulting either from an extra chromosome, or from the deficiency of a chromosome, create an imbalance in the population of fetal DNA molecules in the maternal cell-free plasma DNA that is detectable.

Developing a reliable method for prenatal diagnosis of fetal chromosomal abnormalities has been a long-term goal in reproductive care (Puszyk et al., 2008, Prenat Diagn 28, 1-6). Methods based on obtaining fetal material by amniocentesis or chorionic villus sampling are invasive, and carry a non-negligible risk to the pregnancy even in the hands of skilled clinicians. In current practice, such invasive diagnostic methods are usually employed where there is an indication of an increased chance of a Down's pregnancy, either by reason of maternal age, or through prior screening using biochemical tests or ultrasonography. There is a need for a method of non-invasive prenatal diagnosis (NIPD) that is reliable, applicable in the first trimester, fast in returning a result, and inexpensive.

Progress towards achieving this goal has been made by exploiting the finding that cell-free DNA in the blood plasma of pregnant women includes a component of fetal origin (Lo et al., 1997, Lancet 350, 485-487). The cell-free plasma DNA (referred to hereinafter as ‘plasma DNA’) consists primarily of short DNA molecules (80-200bp) of which typically 5%-20% are of fetal origin, the remainder being maternal (Birch et al., 2005, Clin Chem 51, 312-320; Fan et al., 2010, Clin Chem 56, 1279-1286). The cellular origins of plasma DNA molecules, and the mechanisms by which they enter the blood and are subsequently cleared from the circulation, are poorly understood. However, it is widely believed that the fetal component is largely the result of apoptotic cell death within the placenta (Bianchi, 2004, Placenta 25, S93-S101). The fraction of the plasma DNA molecules that are of fetal origin varies from case to case with substantial individual variation. Superimposed on the individual variation is a general trend towards an increasing fetal component as gestational age increases (Birch et al., 2005, supra; Galbiati et al., 2005, Hum Genet 117, 243-248). The fetal component is readily detectable early in gestation, typically as early as week 8.

In principle, if the cell-free fetal DNA in plasma were undiluted by the maternal component, the extra chromosome that characterises T21 would be expected to cause a 50% excess of DNA molecules derived from that chromosome, by comparison with a normal pregnancy. However, taking a typical value of 10% for the component of cell-free plasma DNA that is of fetal origin, the imbalance that results is expected to be only 5%, or a relative increase in the number of chromosome 21-derived fragments to a value of 1.05 relative to 1.00 for a normal pregnancy. In situations where the fetal component of the plasma DNA is smaller or larger than the 10% value, the imbalance in the number of chromosome 21-derived molecules in the population of molecules in maternal plasma will be correspondingly smaller or larger.

Therefore, the basis of a diagnostic test for T21 is to obtain nucleotide sequence data ('DNA sequencing') for DNA molecules from maternal plasma. Once partial or complete nucleotide sequence information has been obtained from individual DNA molecules, bioinformatic techniques must be applied to assign, most simply by comparison with a reference human genome or genomes, individual molecules to chromosomes from which they originate. In cases of pregnancy involving a fetus with T21, a slight imbalance in the population of molecules is detectable as an excess in the number of chromosome 21-derived molecules over that expected from a normal pregnancy.

In view of the fact that chromosome 21 comprises only a small fraction of the human genome (less than 2%), in order to collect a large enough number from that chromosome for reliable diagnosis, a large number of DNA molecules from maternal plasma must be randomly sampled, sequenced, and assigned bioinformatically to particular chromosomes. The total number of plasma DNA molecules required to be both (1) characterised by nucleotide sequence information derived from them, and then (2) reliably assigned to chromosomal locations, is smaller than that required to sample all or most of the fetal genome, but it is at least several hundred thousand molecules. The minimal number required is a function of the fraction of the plasma DNA that makes up the fetal component of the population of maternal cell-free plasma DNA molecules. Typically, the number is between one million or several million molecules.

The challenge of applying this method is considerable because of the high quantitative accuracy required in counting DNA molecules from particular chromosomal locations. Furthermore, the DNA from maternal plasma is a mixture of genomes within which the fetal component is a small part. This quantitative technical problem is different in nature from identifying mutations at a particular locus within a DNA sample.

Given that some nucleotide sequence data can be obtained for sufficiently large numbers of plasma DNA, and given that bioinformatic methods can be reliably applied to assign a sufficiently large number to their chromosomal origin, statistical methods may be applied to determine the presence or absence of a chromosomal imbalance in the population of plasma DNA molecules with statistical confidence.

This idea of sequencing a random sample of DNA fragments from maternal plasma, but the sample making up only a fraction of the complete genome, is the basis of NIPD methodology described in Fan et al., 2008, Proc Natl Acad Sci U S A 105, 16266-16271 and Chiu et al., 2008, Proc Natl Acad Sci U S A 105, 20458-20463.

An obvious method to utilise sequencing data would be to exclude all fragments outside of a specified range, therefore increasing the fetal fraction in silico. However, this approach would render most of the sequencing data useless and would require a significant increase in the amount of sample processed and sequenced. Thus, digital enrichment could be considered as expensive, inefficient and impractical for use in a routine laboratory environment.

An alternative solution would be to enrich the proportion of the DNA originating from the fetus prior to sequencing. Such enrichment is already typically utilised via size selection methods that remove fragments of approximately 200 bp or larger. Such methods have limited sensitivity and ability to enrich fetal fraction. To date, no method has been described that would allow a highly accurate and precise enrichment of fetal DNA from a biological sample.

There is therefore a need for a highly accurate, non-invasive and simplified method for enriching fetal DNA from a biological sample, in order to detect fetal chromosome abnormalities. Such a method would enhance the performance of non-invasive pre-natal testing for common chromosomal abnormalities, such as Trisomy's 13, 18 and 21. It would also significantly improve the ability to detect much smaller chromosomal abnormalities, such as micro-deletions, where performance is currently very poor relative to the more common chromosomal abnormalities.

### SUMMARY OF THE INVENTION

According to a first aspect of the invention there is provided a method of detecting a fetal chromosomal abnormality which comprises the steps of:
(a) isolating nucleic acids from within a biological sample obtained from a pregnant female subject;
(b1) selecting a nucleic acid fragment size value of between 120 bp and 135 bp for optimal fetal fraction;
(b2) isolating nucleic acid fragments having a size within 20 bp of the fragment size value selected in step (b1);
(c) determining a first number of said fragments which align to a target region of a target chromosome and determining a second number of said fragments which align to one or more target regions within reference chromosomes;
(d) calculating a ratio or difference between the first and second numbers;
(e) determining the presence of a fetal abnormality of said target chromosome based on said ratio or difference.

According to a second aspect of the invention there is provided a method of predicting the gender of a fetus within a pregnant female subject, the method comprising the steps of:
(a) isolating nucleic acids from within a biological sample obtained from a pregnant female subject;
(b1) selecting a nucleic acid fragment size value of between 120 bp and 135 bp for optimal fetal fraction;
(b2) isolating nucleic acid fragments having a size within 20 bp of the fragment size value selected in step (b1);
(c) determining a first number of said fragments which align to a sex chromosome and determining a second number of said fragments which align to one or more reference chromosomes;
(d) calculating a ratio or difference between the first and second numbers;
(e) determining the gender of said fetus based on whether an excess or equivalence of fragments align to an X chromosome compared to said reference chromosome or whether a Y chromosome is present or absent.

### BRIEF DESCRIPTION OF THE FIGURES

FIG. 1: Chromosome 21 ratios at 135 bp target fragment size (±10 bp).

FIG. 2: Fetal fraction estimates at 135 bp target fragment size (±10 bp).

FIG. 3: Chromosome X ratios at 135 bp target fragment size (±10 bp).

FIG. 4: Fragment size profiles at 135 bp target fragment size (±10 bp). and relatively smaller maternal peaks.

FIG. 5: Repeatability Data at 135 bp target fragment size (±10 bp).

FIG. 6: Chromosome 21 ratios at 135 bp target fragment size (±5 bp).

FIG. 7: Chromosome 21 ratios at 135 bp target fragment size (±20 bp).

FIG. 8: Chromosome 21 ratios at 120 bp target fragment size (±10 bp).

FIG. 9: Chromosome 21 ratios at 170 bp target fragment size (±10 bp).

FIGS. 10 and 11: Fetal Fraction estimates at several target fragment sizes and ranges.

FIG. 12: Modelled probability of fragment of a given size being fetal in origin and typical maternal fragment size distribution (10% fetal fraction).

FIG. 13: Graphical Representation Depicting the Probability of a fragment of a given size being fetal.

FIG. 14: Autosome ratio comparison, size-weighted vs. unweighted (chromosome 21).

FIG. 15: Autosome ratio comparison, size-weighted vs. unweighted (chromosome 18).

FIG. 16: Autosome ratio comparison, size-weighted vs. unweighted (chromosome 13).

FIG. 17: Distributions of T21-affected and unaffected sample groups for unweighted and weighted analysis methods.

FIG. 18: Distributions of T18-affected and unaffected sample groups for unweighted and weighted analysis methods.

FIG. 19: Distributions of T13-affected and unaffected sample groups for unweighted and weighted analysis methods.

FIG. 20: Effective fetal fraction at analysis for both the unweighted and size-weighted analysis methods.

FIG. 21: Comparison of effective fetal fraction for trisomy-affected samples at analysis, between unweighted and size-weighted analysis methods.

### DETAILED DESCRIPTION OF THE INVENTION

According to a first aspect of the invention there is provided a method of detecting a fetal chromosomal abnormality which comprises the steps of:
(a) isolating nucleic acids from within a biological sample obtained from a pregnant female subject;
(b1) selecting a nucleic acid fragment size value of between 120 bp and 135 bp for optimal fetal fraction;
(b2) isolating nucleic acid fragments having a size within 20 bp of the fragment size value selected in step (b1);
(c) determining a first number of said fragments which align to a target region of a target chromosome and determining a second number of said fragments which align to one or more target regions within reference chromosomes;
(d) calculating a ratio or difference between the first and second numbers;
(e) determining the presence of a fetal abnormality of said target chromosome based on said ratio or difference.

According to one aspect of the disclosure which may be mentioned there is provided a method of detecting a fetal chromosomal abnormality which comprises the steps of:
(a) isolating nucleic acids from within a biological sample obtained from a pregnant female subject;
(b) isolating nucleic acid fragments having a size from approximately 80 bp to approximately 150 bp;
(c) determining a first number of said fragments which align to a target region of a target chromosome and determining a second number of said fragments which align to one or more target regions within reference chromosomes;
(d) calculating a ratio or difference between the first and second numbers;
(e) determining the presence of a fetal abnormality of said target chromosome based on said ratio or difference.

In detecting fetal chromosomal abnormalities, it is important to ensure, as much as possible, that false results are not determined. In particular, it is particularly desired to reduce a probability of a false negative result being determined. However, it is also important to ensure that data is efficiently used and that positive and accurate results are generated in an acceptable number of cases or tests. A test result should, ideally, be declared where possible, rather than a test indicating that the result is unreliable due to one or more parameters associated with the test.

The proportion of cell free DNA originating from the fetus is a critical parameter for the detection of chromosomal abnormalities in fetal samples. A minimum proportion of DNA, in combination with other factors, is required for accurate detection. In particular, smaller chromosomal abnormalities such as microdeletions require a larger proportion of DNA originating from the fetus in order to be detectable. The inventors of the present invention have surprisingly identified that enriching fetal material to a fragment size from approximately 80 bp to approximately 150 bp has significantly improved the accuracy and performance of such tests as supported by the data presented herein. In addition, the method of the invention generates a significantly lower amount of sequencing data which therefore results in a more time efficient and cost efficient fetal chromosomal abnormality detection method.

A number of publications since 2004 (i.e. EP 2 728 014) have indicated that maternal and fetal cell-free DNA fragments in maternal circulation possess differing size distributions. Additionally, it has been noted in the literature that it might be possible to exploit this difference in analysis for non-invasive prenatal testing (NIPT). However, to date no direct, deterministic theoretical relationship has been published in the literature between the size of a DNA fragment drawn from a mixed maternal/fetal cfDNA sample and the probability that said fragment is fetal or maternal in origin. Such a model is required to be able to determine optimal size ranges for use in a size selection-based process to enrich effectively the fraction of fetal DNA in samples to be subject to NIPT analysis, and is particularly useful to inform the choice of ranges where instrument-related practical considerations on allowed size ranges come into play.

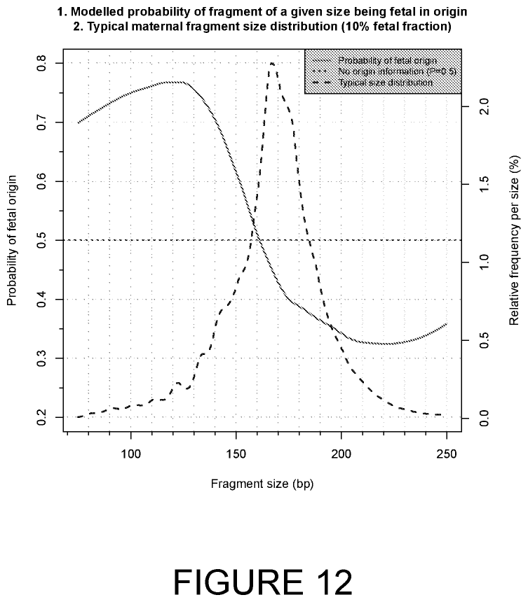

To support the present invention, FIG. 12 shows such a model constructed by the inventors employing previously published size distribution data. The solid line represents the modelled probability that a fragment of a given size is fetal in origin, and for reference the dashed line represents the total distribution of fragments by size for a sample with a total fetal fraction of 10% (that is, independently of size, the total probability of any given fragment being from the fetus is 0.1).

The model depicted by the solid line in FIG. 12 may be used directly to inform the choice of size fraction to enrich an NIPT sample optimally for fetal DNA, by choosing a size range which maximises large probability values as far as possible. Independently of any other considerations on size range, a typical optimal range could for example be 120±10 bp (to include only the peak of probability), however for practical implementation purposes alternative ranges may be chosen which still enrich effectively for fetal DNA, such as:
135±10 bp, if the particular size selection technology in use can operate only, or operate best, within certain size parameters; and
80-150 bp (i.e. 115±35 bp), for example if the technology in use requires a large size range.

The probability model of FIG. 12 has in fact been constructed for the case that maternal and fetal fragments overall (i.e. without regard to size) are equally likely to occur in a sample. This overall balance of course varies in practical samples, with the fetal fraction ranging between approximately 3% and 25%, however the relative concentration of maternal and fetal fragments in response to size will still follow the same profile independent of overall fetal fraction.

Furthermore, the method of the invention allows for a significant improvement in the efficiency of the resultant sequencing. For example, the increase of fetal fraction percentage prior to analysis enables a significant reduction in the amount of data required for an accurate detection of a fetal chromosomal abnormality.

It will be appreciated that the term “within 20 bp” refers to +/−20 bp, i.e. a total range of nucleic acid fragments of 40 bp. In one embodiment, isolation in step (b2) is of nucleic acid fragments within 10 bp (i.e. a total range of fragments of 20 bp). In a further embodiment, isolation in step (b2) is of nucleic acid fragments within 5 bp (i.e. a total range of fragments of 10 bp).

The inventors have identified that any 40 bp “window” from 100 bp to 155 bp provides optimal results as has been shown in the data presented and discussed herein. Thus, prior to analysis, an arbitrary value is chosen from 120 bp to 135 bp (step (b1) as described herein). The inventors have surprisingly found that any value between these ranges provides the optimal fetal fraction as the majority of the fetal chromosomal fragments will be of this size. In one embodiment, the value selected in step (b1) is 120 bp, or 121 bp, or 122 bp, or 123 bp, or 124 bp, or 125 bp, or 126 bp, or 127 bp, or 128 bp, or 129 bp, or 130 bp, or 131 bp, or 132 bp, or 133 bp, or 134 bp, or 135 bp.

One key aspect of the invention is acknowledgement that the user must not only select the above mentioned arbitrary value in step (b1) but also then ensure that a range of sizes closely approximating this size are then analysed. This is important because if 125 bp is selected as the arbitrary value in step (b1) and only fragments with this size were identified then the number of reads would not be sufficient to generate a significant and most crucially an accurate enough result. Therefore, analysing all fragments within 20 bp or 10 bp or 5 bp (i.e. a total nucleic acid fragment range of 40 bp or 20 bp or 10 bp) of the size selected in step (b1) will provide a larger number of mostly fetal chromosomal fragments to significantly improve the sensitivity and accuracy of the result. Thus, to summarise there is a synergy between steps (b1) and (b2) such that step (b1) provides the optimal size value for maximal fetal concentration and the range in step (b2) maximises the total number of fetal fragments.

### Fetal Chromosomal Abnormalities

It will be appreciated that references herein to “fetal chromosomal abnormality” refer to any genetic variation within a fetal chromosome and includes any variation in the native, non-mutant or wild type genetic code of said fetus. Examples of such genetic variations include: aneuploidies, duplications, translocations, mutations (e.g. point mutations), substitutions, deletions, single nucleotide polymorphisms (SNPs), chromosome abnormalities, Copy Number Variation (CNV), epigenetic changes and DNA inversions.

References herein to the term “single-nucleotide polymorphism (SNP)” is intended to refer to DNA sequence variation occurring when a single nucleotide in a given gene differs between members of a species or between paired chromosomes in an individual.

In one embodiment the genetic variation is a functional mutation i.e. one which is causative of a clinically relevant fetal disease or disorder. Examples of such a disease or disorder include thalassemia and cystic fibrosis, in addition to fragment length disorders, such as fragile X syndrome. Mutations may be functional in that they affect amino acid encoding, or by disruption of regulatory elements (e.g., which may regulate gene expression, or by disruption of sequences - which may be exonic or intronic - involved in regulation of splicing).

Examples of suitable fetal chromosomal abnormalities which the invention finds utility in detecting include: Down's Syndrome (Trisomy 21), Edward's Syndrome (Trisomy 18), Patau syndrome (Trisomy 13), Trisomy 9, Warkany syndrome (Trisomy 8), Cat Eye Syndrome (4 copies of chromosome 22), Trisomy 22, and Trisomy 16.

Additionally, or alternatively, the detection of an abnormality in a gene, chromosome, or part of a chromosome, copy number may comprise the detection of and/or diagnosis of a condition selected from the group comprising Wolf-Hirschhorn syndrome (4p-), Cri du chat syndrome (5p-), Williams-Beuren syndrome (7-), Jacobsen Syndrome (11-), Miller-Dieker syndrome (17-), Smith-Magenis Syndrome (17-), 22g1 1.2 deletion syndrome (also known as Velocardiofacial Syndrome, DiGeorge Syndrome, conotruncal anomaly face syndrome, Congenital Thymic Aplasia, and Strong Syndrome), Angelman syndrome (15-), and Prader-Willi syndrome (15-).

Additionally, or alternatively, the detection of an abnormality in the chromosome copy number may comprise the detection of and/or diagnosis of a condition selected from the group comprising Turner syndrome (Ullrich-Turner syndrome or monosomy X), Klinefelter's syndrome, 47,XXY or XXY syndrome, 48,XXYY syndrome, 49,XXXXY Syndrome, Triple X syndrome, XXXX syndrome (also called tetrasomy X, quadruple X, or 48,XXXX), XXXXX syndrome (also called pentasomy X or 49,XXXXX) and XYY syndrome.

In one embodiment, the target chromosome is chromosome 13, chromosome 18, chromosome 21, the X chromosome or the Y chromosome.

In one embodiment, the fetal chromosomal abnormality is a fetal chromosomal aneuploidy. In a further embodiment, the fetal chromosomal aneuploidy is trisomy 13, trisomy 18 or trisomy 21. In a yet further embodiment, the fetal chromosomal aneuploidy is trisomy 21 (Down's syndrome). In this embodiment, the skilled worker in the field will readily understand that the methodology of the invention can be applied to diagnosing cases where the fetus carries a substantial part of chromosome 21 rather than an entire chromosome.

In one embodiment, the fetal chromosomal abnormality is a chromosomal insertion or a deletion, for example of up to 1 Mb, up to 5 Mb, up to 10 Mb or up to 20 Mb or greater than 20 Mb.

### Sample Extraction

It will be appreciated that samples may be obtained from the pregnant female subject in accordance with routine procedures. In one embodiment, the biological sample is maternal blood, plasma, serum, urine or saliva. In a further embodiment the biological sample is maternal plasma.

The step of obtaining maternal plasma will typically involve a 5-20 ml blood sample (typically a peripheral blood sample) being withdrawn from the pregnant female subject (typically by venipuncture). Obtaining such a sample is therefore characterised as noninvasive of the fetal space, and is minimally invasive for the mother. Blood plasma is prepared by conventional means after removal of cellular material by centrifugation (Maron et al., 2007, Methods Mol Med 132, 51-63).

DNA is extracted from the maternal plasma by conventional methodology which is unbiased with respect to the nucleotide sequences of the plasma DNA (Maron et al., 2007, supra). The population of plasma DNA molecules will typically comprise a fraction that is of fetal origin, and a fraction of maternal origin.

Isolation of Nucleic Acids From Within a Biological Sample

In one embodiment, the step of isolating in step (a) comprises the preparation of a library of nucleic acid fragments. It will be appreciated that the steps of isolating, fragmenting and library preparation may be conducted in accordance with routine procedures well known to the skilled person. In a further embodiment, library preparation comprises the sequential steps of DNA end repair, adaptor ligation, clean up and PCR. Full experimental details of how a suitable nucleic acid library may be prepared are described in the methods section herein, in particular steps 1-49.

### Enrichment

In one embodiment, isolation step (b2) comprises enrichment for nucleic acid fragments having a size within 10 bp of the fragment size value selected in step (b1), such as within 5 bp of the fragment size value selected in step (b1).

In one embodiment of the disclosure, the isolation step (b2) comprises enrichment for nucleic acid fragments having a size of 115 ±35 bp (i.e. 80-150 bp), such as 115 ±30 bp, 115±25 bp, 115±20 bp, 115±15 bp, 115±10 bp, 120±10 bp, 110 ±10 bp, 135±10 bp, 140±10 bp, 115±5 bp or 115 bp.

In a further embodiment, isolation step (b2) comprises enrichment for nucleic acid fragments having a size of 120±10 bp, 110±10 bp, 135±10 bp, 140±10 bp, 115±5 bp or 115 bp.

It will be appreciated that such enrichment step may be conducted in accordance with routine procedures well known to the skilled person. In one embodiment, isolation step (b2) comprises enrichment using size selection. In a further embodiment, isolation step (b2) comprises enrichment using gel based size selection. In a further embodiment, isolation step (b2) comprises enrichment using automated gel based size selection.

One such example of automated gel based size selection includes the Ranger Technology ^{TM} from Coastal Genomics.

The Ranger Technology™ makes use of an isolated box which creates a dark environment to prevent the effect of light on analysis. Currently the cassettes are of a proprietary size rather than SSID to match other automation footprints. Cassettes contain formed agarose gel with 12 channels for use.

Samples are processed as per standard electrophoresis whereby the charge generated at the ends of the cassette causes movement and separation of DNA fragments depending on size (and as such charge). No ladder is used but a mixture of a lower and upper markers are provided to ensure that sizing can be performed within sample. Outputs may be displayed in electropherogram or gel image formats.

Samples of the required size will be processed out into the solution contained within the well identified for removal, it is here that the entire volume will be removed and replenished as many times as informed by the Ranger software.

The Ranger Technology™ takes images of the gel throughout the migration process in blue and red lights that provide visibility to sample and markers based on the associated dyes that become excited in the presence of that light (each having their own fluorescence with which to reduce incorrect results associated with incorrect marker identification). Full details of the Ranger Technology™ may be seen at http://coastalgenomics.com/.

In an alternative embodiment which may be mentioned, the method of the invention may involve a low melting point agarose based method. This embodiment requires DNA fragments from a sample to be run on a suitable agarose gel, then excised from the gel using a manual means (e.g., a fine band of the gel cut using a disposable knife).

In an alternative embodiment which may be mentioned, the method of the invention may involve a bead based size selection method instead of gel based size selection. This embodiment requires a bead based method that selects DNA fragments based on their size in base pairs, to a very high degree of accuracy and precision.

In an alternative embodiment which may be mentioned, the method of the invention may involve a PCR based method. This embodiment requires PCR to be setup whereby fragments longer than a specified base pair length are unable to amplify (or amplify with much reduced efficiency).

In an alternative embodiment which may be mentioned, the method of the invention may involve an enzyme digestion based method. This embodiment requires the use of enzymes to digest (or preferentially digest) DNA fragments above a specified length.

Several of the embodiments hereinbefore utilise a physical separation of the DNA fragments based on their size which require a subsequent step to purify the fragments of the desired size away from the unwanted fragments. This can be achieved by constructing a standard curve based on the behaviour of DNA fragments of known size and then using this standard curve to isolate the region of the size separated fragments which contain the fragments of interest.

Alternatively one or more labelled molecules can be constructed which have similar behaviour to the DNA fragments of interest when subjected to the size separation method. These labelled molecules can be mixed with the DNA to be size separated and then, following the separation process, the region which contains the labelled fragment will also contain the DNA fragments of interest.

Following the separation it is necessary to isolate the set of size separated DNA fragments of interest.

There are many different approaches to isolating DNA fragments of the required size range. In one method a standard curve is constructed using DNA fragments of known size which is then used to isolate the DNA fragments of interest.

### Fragment Alignment

Step (c) of the method of the invention conducts an alignment or matching analysis. Such an analysis will initially require measurement of the presence of one or more target sequences within the fragments isolated in step (b2) or alternatively sequencing of said fragments. Thus, in one embodiment, step (c) initially comprises sequencing the fragments isolated in step (b2) or subjecting said fragments to digital PCR or SNP based methodology prior to alignment.

### Sequencing

In a further embodiment, step (c) initially comprises sequencing the fragments isolated in step (b2). It will be appreciated by the skilled person that the invention is not limited to any particular technique for sequencing the enriched fragments and obtaining the sequence data. In one embodiment, the sequence data is obtained by a sequencing platform which comprises use of a polymerase chain reaction. In a further embodiment, the sequence data is obtained using a next generation sequencing platform. Such sequencing platforms have been extensively discussed and reviewed in: Loman et al (2012) Nature Biotechnology 30(5), 434-439; Quail et al (2012) BMC Genomics 13, 341; Liu et al (2012) Journal of Biomedicine and Biotechnology 2012, 1-11; and Meldrum et al (2011) Clin Biochem Rev. 32(4): 177-195; the sequencing platforms of which are herein incorporated by reference.

Examples of suitable next generation sequencing platforms include: Roche 454 (i.e. Roche 454 GS FLX), Applied Biosystems' SOLiD system (i.e. SOLiDv4), Illumina's GAllx, HiSeq 2000 and MiSeq sequencers, Life Technologies' Ion Torrent semiconductor-based sequencing instruments, Pacific Biosciences' PacBio RS and Sanger's 3730xl.

Each of Roche's 454 platforms employ pyrosequencing, whereby chemiluminescent signal indicates base incorporation and the intensity of signal correlates to the number of bases incorporated through homopolymer reads.

In one embodiment, the enriched fragments are sequenced by a sequencing platform which comprises use of semiconductor-based sequencing methodology. The virtues of semiconductor-based sequencing methodology are that the instrument, chips and reagents are very cheap to manufacture, the sequencing process is fast (although off-set by emPCR) and the system is scalable, although this may be somewhat restricted by the bead size used for emPCR.

In one embodiment, the enriched fragments are sequenced by a sequencing platform which comprises use of sequencing-by-synthesis. Illumina's sequencing-by-synthesis (SBS) technology is currently a successful and widely-adopted next-generation sequencing platform worldwide. TruSeq technology supports massively-parallel sequencing using a proprietary reversible terminator-based method that enables detection of single bases as they are incorporated into growing DNA strands. A fluorescently-labeled terminator is imaged as each dNTP is added and then cleaved to allow incorporation of the next base. Since all four reversible terminator-bound dNTPs are present during each sequencing cycle, natural competition minimizes incorporation bias.

In one embodiment, the enriched fragments are sequenced by a sequencing platform which comprises use of nanopore-based sequencing methodology. In a further embodiment, the nanopore-based methodology comprises use of organic-type nanopores which mimic the situation of the cell membrane and protein channels in living cells, such as in the technology used by Oxford Nanopore Technologies (e.g. Branton D, Bayley H, et al (2008). Nature Biotechnology 26 (10), 1146-1153). In a yet further embodiment, the nanopore-based methodology comprises use of a nanopore constructed from a metal, polymer or plastic material.

In one embodiment, the next generation sequencing platform is selected from Life Technologies' Ion Torrent platform or Illumina's MiSeq. The next generation sequencing platforms of this embodiment are both small in size and feature fast turnover rates but provide limited data throughput.

In a further embodiment, the next generation sequencing platform is a personal genome machine (PGM) which is Life Technologies' Ion Torrent Personal Genome Machine (Ion

Torrent PGM). The Ion Torrent device uses a strategy similar to sequencing-by-synthesis (SBS) but detects signal by the release of hydrogen ions resulting from the activity of DNA polymerase during nucleotide incorporation. In essence, the Ion Torrent chip is a very sensitive pH meter. Each ion chip contains millions of ion-sensitive field-effect transistor (ISFET) sensors that allow parallel detection of multiple sequencing reactions. The use of ISFET devices is well known to the person skilled in the art and is well within the scope of technology which may be used to obtain the sequence data required by the methods of the invention (Prodromakis et al (2010) IEEE Electron Device Letters 31(9), 1053-1055; Purushothaman et al (2006) Sensors and Actuators B 114, 964-968; Toumazou and Cass (2007) Phil. Trans. R. Soc. B, 362, 1321-1328; WO 2008/107014 (DNA Electronics Ltd); WO 2003/073088 (Toumazou); US 2010/0159461 (DNA Electronics Ltd); the sequencing methodology of each are herein incorporated by reference).

In one embodiment, the enriched fragments are sequenced by a sequencing platform which comprises use of release of ions, such as hydrogen ions. This embodiment provides a number of key advantages. For example, the Ion Torrent PGM is described in Quail et al (2012; supra) as the most inexpensive personal genome machines on the market (i.e. approx. $80,000). Furthermore, Loman et al (2012; supra) describes the Ion Torrent PGM as producing the fastest throughput (80-100 Mb/h) and the shortest run time (˜3 h).

It will be appreciated that later generations of the Ion Torrent device may also find utility in the invention, for example in one embodiment, the sequence data is obtained by multiplex capable iterations based upon the Life Technologies' Ion Torrent platform, such as an Ion Proton with a PI or PII Chip, and further derivative devices and components thereof.

### Digital PCR

In a further embodiment, step (c) initially comprises subjecting the fragments isolated in step (b2) to digital PCR. It will be appreciated by the skilled person that the invention is not limited to any particular technique for digital PCR of the enriched fragments and obtaining the data. The present invention lends itself particularly well to the use of digital PCR as a fragment analysis method because digital PCR works optimally when the fetal fraction is at least 20% and the present invention provides methodology capable of providing such levels of fetal fraction. Suitable methodology of how digital PCR may be performed on maternal plasma samples is described in EP 1 981 995. Examples of suitable digital PCR systems include: digital PCR system selected from: Quant studio digital PCR system (ThermoFisher) and RainDrop Plus digital PCR system (RainDance technologies).

### Alignment Analysis

Such a matching analysis typically involves a bioinformatic analysis which is performed using suitable software and allocates hits for each fragment of a given chromosome (i.e. a target or reference chromosome) based on whether said fragment aligns with or is deemed to have originated from said chromosome.

In one embodiment, the alignment is conducted using IONA® software (Premaitha Helath plc), Bowtie2 or BWA-SW (Li and Durbin (2010) Bioinformatics, Epub) alignment software or alignment software employing Maximal Exact Matching techniques, such as BWA-MEM (lh3lh3.users.sourceforge.net/download/mem-poster.pdf) or CUSHAW2 (http://cushaw2.sourceforge.net/) software. In a further embodiment, the alignment is conducted using Bowtie2 software. In a yet further embodiment, the Bowtie2 software is Bowtie2 2.0.0-beta7.

In an alternative embodiment, the alignment is conducted using alignment software employing Maximal Exact Matching (MEM) techniques, such as BWA-MEM (lh3lh3.users.sourceforge.net/download/mem-poster.pdf) or CUSHAW2 http://cushaw2.sourceforge.net/) software. The MEM algorithms are believed to have the advantage of providing greater accuracy

It will be appreciated that for maximum accuracy, the sequences should be mapped or aligned to a unique chromosomal location. For example, if a fragment maps to both the target region of a target chromosome and a further chromosome then it should be eliminated from the analysis because it cannot be deemed to be uniquely aligned to the target region of a target chromosome. In one embodiment, the method additionally comprises the step of collapsing duplicate reads from the sequence data obtained prior to alignment step (c).

Thus, in one embodiment, step (c) comprises determining a first number of said fragments which uniquely align to a target region of a target chromosome and determining a second number of said fragments which uniquely align to one or more target regions within reference chromosomes.

It will be appreciated that references herein to “target region” refer to a portion or all of said target and/or reference chromosomes.

In one embodiment, the target chromosome is a region within a chromosome and the reference chromosome is a region within the same chromosome as the target chromosome.

In one embodiment, the method additionally comprises enrichment of the sample for the genomic region suspected to contain the fetal chromosomal abnormality. Such an embodiment will typically make use of a process of selection through a hybridisation based technique and will allow the pre-selection to either retain or remove pre-selected target sequences prior to sequencing.

For the mapping of sequences to unique chromosomal locations, the indel/mismatch cost weighting must be parameterised to low in this analysis. With these pre-conditions, non-stringent fragment-length matches are determined. Using this bioinformatic approach, typically about 95% of sample reads are mapped to the genome. Reads are only counted as assigned to a chromosomal location if they match to a unique position in the genome, typically bringing the proportion of sample reads uniquely matched and subsequently counted for the chromosomal assignments to about 50%.

In one embodiment, the alignment is conducted with respect to a whole chromosome, for example, the analysis would therefore comprise detecting an excess of a given chromosome. In an alternative embodiment, the alignment is conducted with respect to a part of said chromosome, for example, matches will be analysed solely with respect to a particular pre-determined region of a chromosome. It is believed that this embodiment of the invention provides a more sensitive matching technique by virtue of targeting a specific region of a chromosome.

### Size Weighting

In one embodiments of any of the methods described herein (i.e. the abnormality detection and gender prediction methods) additional size weighting steps may be conducted. Thus, in one embodiment, the method additionally comprises the steps of:
(i) size-weighting each fragment which aligns to a target region of a target chromosome by calculating the probability (w) of each fragment size (s) being fetal in origin;
(ii) size-weighting each fragment which aligns to one or more target regions within one or more reference chromosomes by calculating the probability (w) of each fragment size (s) being fetal in origin;
(iii) calculating a total target weighted count (Nc_{target}) by summing the values obtained in step (i);
(iv) calculating a total reference weighted count (Nc) by summing the values obtained in step (ii);
(v) calculating a ratio or difference between the Nc_{target} and Nc values obtained in steps (iii) and (iv); and
(vi) determining the presence of a fetal abnormality of said target chromosome based on said ratio or difference.

The size distribution of cell-free DNA fragments originating from the placenta of a pregnant woman (and hence in the vast majority of cases reflecting the karyotype of the fetus or fetuses) is known to follow a significantly different profile from that of fragments originating from the pregnant woman herself (i.e. from tissues other than the placenta). Most fundamentally, fetal DNA fragments are found to be shorter on average than maternal DNA fragments.

In an analysis focussed on the genome of the fetus the difference in size distribution may be exploited to improve sensitivity by preferentially selecting sequenced fragments which are more likely to be fetal. Previously known bioinformatics approaches involve passing only fragments in a given (shorter) size range for further analysis. However, although this naïve approach may result in a relative enrichment for fetal DNA in the analysis, it also results in the loss of the majority of the fragments being counted, and therefore significantly more uncertainty in the analysis; this then offsets much of the sensitivity benefit of enriching for fetal DNA.

The inventors have developed an alternative approach which makes better use of all fragments analysed. This utilises the known differences in fragment size profiles between fetal and maternal DNA molecules to weight (i.e. prioritise) fragments preferentially in the analysis if these have a higher probability of reflecting the karyotype of the fetus than that of the mother, and conversely to de-emphasise contributions from fragments which have a higher probability of originating from maternal tissue other than the placenta.

Importantly, rather than discarding fragments from counting that are outside a target size range, this new analysis continues to take account of all fragments in the counting process. It therefore retains the statistical power afforded by counting a large proportion of DNA fragments, while simultaneously reducing the dilutive effect of fragments originating from the euploid mother.

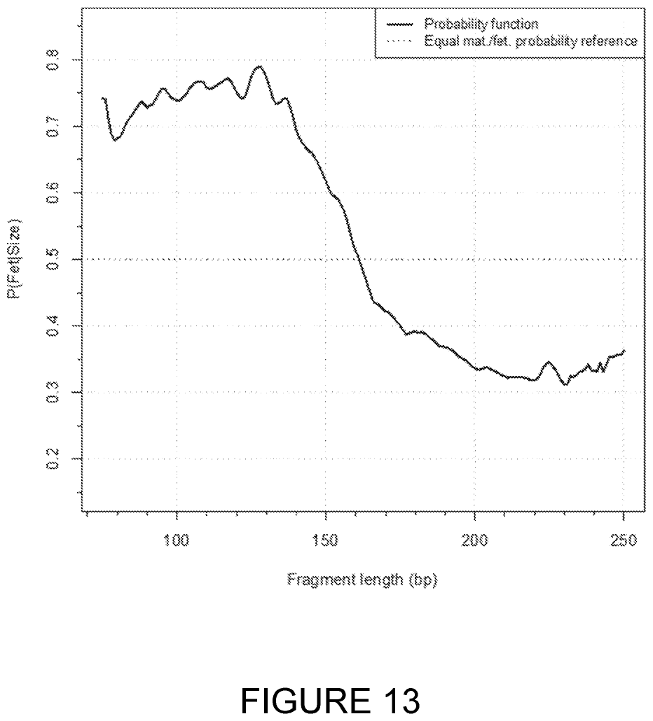

The data provided in FIG. 13 and Table A represents the probability that a cell-free DNA fragment drawn from maternal plasma is fetal in origin, as a function of the size of the fragment. This relationship has been calculated from data expressing the relative frequencies of fragments known to be either fetal or maternal in origin (Chandrananda et al (2015) BMC medical genomics 8.1: 29).

An overview of the bioinformatics approach taken according to the invention is as follows. Prior methodology generate a fragment count value N_{c} for each chromosome c, in which each fragment contributes a value of 1. N_{c} is therefore simply the number of fragments found to map to chromosome c.

In the improved method of the invention which makes use of size-weighting of each fragment each fragment instead contributes a value of w[s], where w is a weighting function and s is the size of the fragment in nucleotides, which is determined as part of the sequencing process. The weighting function w used here is the probability that a fragment is fetal in origin, as plotted in FIG. 13 and extrapolated in the specific probability (w) values shown in Table A. The total weighted count, N_{c}, assigned to a chromosome c then results from summing all of the w[s] values for all fragments found to align against that chromosome.

The total weighted count, N_{c}, resulting from summing all of the w[s] values for all fragments found to align against the target chromosome c_{target} is known herein as N_{c}_{_{target}}. Thus, in one embodiment, the step of calculating the probability (w) of each fragment size (s) being fetal in origin in steps (i) and (ii) comprises identifying the size (s) of each aligned fragment and allocating a w value for said fragment based on the values presented in Table A.

In one embodiment, the N_{c}_{_{target}} value is subjected to a GC correction step (as in prior methodology) and a normalised measure of the presence of fragments from this chromosome in the sample is calculated; this is done for a target chromosome c_{target} by forming a proportion of the fragments counted against all autosomes (the proportion is relative to the sum of the N_{c} values calculated for all autosomal chromsomes; these N_{c} values have all also been subject to a GC correction step).

Calculation of the ratio i.e. ‘autosome ratio’ in step (v) is referred to as calculating the R_{c}_{_{target}} value:

$${R}_{{c}_{target}} = \frac{{N}_{{c}_{target}}}{{\sum }_{c = 1}^{22} {N}_{c}} .$$

This autosome ratio is then used as input to a statistical model, which estimates the probability of trisomy to produce the final test result calculated in step (vi).

TABLE A
Size-Weighting Values of Each Fragment

| Fragment Size in base pairs (s) | Prob(Fetal\|Size) (w) |
|---|---|
| 75 | 0.742 |
| 76 | 0.740 |
| 77 | 0.710 |
| 78 | 0.688 |
| 79 | 0.679 |
| 80 | 0.682 |
| 81 | 0.686 |
| 82 | 0.694 |
| 83 | 0.705 |
| 84 | 0.712 |
| 85 | 0.717 |
| 86 | 0.724 |
| 87 | 0.731 |
| 88 | 0.737 |
| 89 | 0.733 |
| 90 | 0.728 |
| 91 | 0.732 |
| 92 | 0.734 |
| 93 | 0.742 |
| 94 | 0.750 |
| 95 | 0.757 |
| 96 | 0.756 |
| 97 | 0.749 |
| 98 | 0.744 |
| 99 | 0.741 |
| 100 | 0.738 |
| 101 | 0.739 |
| 102 | 0.744 |
| 103 | 0.748 |
| 104 | 0.755 |
| 105 | 0.761 |
| 106 | 0.766 |
| 107 | 0.767 |
| 108 | 0.768 |
| 109 | 0.765 |
| 110 | 0.758 |
| 111 | 0.756 |
| 112 | 0.758 |
| 113 | 0.761 |
| 114 | 0.764 |
| 115 | 0.766 |
| 116 | 0.770 |
| 117 | 0.772 |
| 118 | 0.769 |
| 119 | 0.760 |
| 120 | 0.751 |
| 121 | 0.744 |
| 122 | 0.742 |
| 123 | 0.747 |
| 124 | 0.761 |
| 125 | 0.776 |
| 126 | 0.786 |
| 127 | 0.788 |
| 128 | 0.790 |
| 129 | 0.783 |
| 130 | 0.773 |
| 131 | 0.760 |
| 132 | 0.745 |
| 133 | 0.735 |
| 134 | 0.734 |
| 135 | 0.738 |
| 136 | 0.743 |
| 137 | 0.740 |
| 138 | 0.728 |
| 139 | 0.713 |
| 140 | 0.695 |
| 141 | 0.683 |
| 142 | 0.675 |
| 143 | 0.670 |
| 144 | 0.664 |
| 145 | 0.661 |
| 146 | 0.655 |
| 147 | 0.647 |
| 148 | 0.637 |
| 149 | 0.627 |
| 150 | 0.617 |
| 151 | 0.605 |
| 152 | 0.598 |
| 153 | 0.594 |
| 154 | 0.591 |
| 155 | 0.582 |
| 156 | 0.573 |
| 157 | 0.559 |
| 158 | 0.542 |
| 159 | 0.524 |
| 160 | 0.513 |
| 161 | 0.502 |
| 162 | 0.489 |
| 163 | 0.475 |
| 164 | 0.460 |
| 165 | 0.447 |
| 166 | 0.436 |
| 167 | 0.434 |
| 168 | 0.431 |
| 169 | 0.427 |
| 170 | 0.422 |
| 171 | 0.421 |
| 172 | 0.416 |
| 173 | 0.411 |
| 174 | 0.405 |
| 175 | 0.399 |
| 176 | 0.393 |
| 177 | 0.388 |
| 178 | 0.390 |
| 179 | 0.391 |
| 180 | 0.391 |
| 181 | 0.390 |
| 182 | 0.391 |
| 183 | 0.389 |
| 184 | 0.385 |
| 185 | 0.382 |
| 186 | 0.378 |
| 187 | 0.374 |
| 188 | 0.369 |
| 189 | 0.369 |
| 190 | 0.369 |
| 191 | 0.367 |
| 192 | 0.364 |
| 193 | 0.362 |
| 194 | 0.357 |
| 195 | 0.353 |
| 196 | 0.351 |
| 197 | 0.348 |
| 198 | 0.344 |
| 199 | 0.340 |
| 200 | 0.336 |
| 201 | 0.334 |
| 202 | 0.335 |
| 203 | 0.337 |
| 204 | 0.338 |
| 205 | 0.336 |
| 206 | 0.333 |
| 207 | 0.331 |
| 208 | 0.329 |
| 209 | 0.326 |
| 210 | 0.324 |
| 211 | 0.323 |
| 212 | 0.323 |
| 213 | 0.323 |
| 214 | 0.324 |
| 215 | 0.324 |
| 216 | 0.323 |
| 217 | 0.322 |
| 218 | 0.321 |
| 219 | 0.318 |
| 220 | 0.319 |
| 221 | 0.321 |
| 222 | 0.328 |
| 223 | 0.339 |
| 224 | 0.344 |
| 225 | 0.346 |
| 226 | 0.340 |
| 227 | 0.335 |
| 228 | 0.326 |
| 229 | 0.317 |
| 230 | 0.313 |
| 231 | 0.313 |
| 232 | 0.324 |
| 233 | 0.323 |
| 234 | 0.327 |
| 235 | 0.330 |
| 236 | 0.332 |
| 237 | 0.335 |
| 238 | 0.342 |
| 239 | 0.334 |
| 240 | 0.333 |
| 241 | 0.331 |
| 242 | 0.346 |
| 243 | 0.331 |
| 244 | 0.341 |
| 245 | 0.353 |
| 246 | 0.353 |
| 247 | 0.355 |
| 248 | 0.357 |
| 249 | 0.357 |
| 250 | 0.363 |

The size distribution of cell-free DNA fragments originating from the placenta or placentae of a pregnant woman (and hence in the vast majority of cases reflecting the karyotype of the fetus or fetuses) is known to follow a significantly different profile from that of fragments originating from the pregnant woman herself (i.e. from tissues other than the placenta). This phenomenon is exploited by the method of the invention.

It is also possible to utilise these known differences in fragment size profiles to weight fragments preferentially for chromosome ratio computation if these have a higher probability of reflecting the karyotype of the fetus than that of the mother, and conversely to de-emphasise contributions from fragments which have a higher probability of originating from maternal tissue other than the placenta.

One specific, non-limiting method of size-weighting is described as follows:

### Method

The following static data items are required to be included in configuration data for the software:
A fragment size count weighting map, w[s], where s is an integer fragment size value in the inclusive range CountWeightFragSizeMin (sₘᵢₙ) to CountWeightFragSizeMax (sₘₐₓ). The range limits are also required to be specified as part of configuration data. The weighting map values are to be stored in a real number type with precision equivalent to or greater than that of ‘single precision’ format (as defined by IEEE-754: 1985).
A size-weighting enable/disable flag (boolean) of each fragment, CountWeightFragSizeEnable.
A ‘missing fragment size’ action (enumerated), CountWeightFragSizeMissingAction. This may take on the values Ignore or Integrate.

The method proceeds as follows for any unique fragment alignment event, generating a count increment u.

If CountWeightFragSizeEnable is false, no weighting operation shall take place, i.e.
u=1.

If CountWeightFragSizeEnable is true, the count increment is generated with reference to the fragment size and weighting map, as follows.

Firstly, per-count accumulated values are initialised:
W←0
N_{contrib}←0.

Then, for each read in the set of reads which were collapsed to a single counting event (i.e. were considered to be duplicates following alignment),
1. An attempt is made to obtain a fragment size measurement (s). The precise manner of obtaining this depends on the particular sequencing platform in use, but might for example be: the contents of the ZA tag that may be stored with the read in the Basecaller BAM file in the case of Thermo Fisher Ion Torrent family sequencing systems employing a single-ended sequencing protocol, or a value extracted from a sequence alignment step if a paired-end sequencing protocol has been used. The skilled person will appreciate that a number of other possible mechanisms may exist to obtain a size value, the method being tailored to the sequencing platform and protocol in use. If a value for s can be obtained, an attempt is made to look it up in the weight map: if the value s is within the weight map bounds (i.e. sₘᵢₙ≤s≤sₘₐₓ), per-count accumulated values are updated as follows:
W←W+w[s]
N_{contrib}←N_{contrib}+1.

Alternatively if s<sₘᵢₙ or s>sₘₐₓ, no update takes place to accumulated values.
2. If no ZA tag was associated with the read, the action taken depends on the value of CountWeightFragSizeMissingAction, as follows:
   a. If CountWeightFragSizeMissingAction has the value Integrate, the sequencing read length for the fragment (l) is used as a lower bound on a size range, with the upper bound for the range being CountWeightFragSizeMax. An integrated weight is then calculated by averaging over the size range, and the accumulated values updated:

$$W ← W + \frac{1}{1 + {s}_{ma x} - l} {\sum }_{s = l}^{{s}_{ma x}} w [ s ]$$

$$N ← {N}_{contrib} + 1.$$

b. If CountWeightFragSizeMissingAction has the value Ignore, the fragment is discarded and no updates take place to accumulated values.

When all reads contributing to the counting event have been considered, the count increment is then generated as follows:

$$u = \frac{W}{{N}_{contrib}} .$$

The value ultimately determined for u is finally added to the accumulated aligned fragment count (N_{c}) for the chromosome against which it was found to align. Accumulated, weighted aligned fragment counts determined in this way are subject to correction according to GC content, as in prior methodology, and the corrected values then used in computation of autosome and other chromosome ratios for input to trisomy likelihood models (Rᵢₙ values), fetal fraction estimation (Rₓ) and sex determination (Rₓ and optionally also R_{Y}).

There is one exception to this: A chromosome ratio that is to be used as part of the Run Control validity check should not be subject to weighting according to fragment size (but should still be subject to GC correction).

Where a count of autosome or chromosome fragments is to be used for any purpose other than in the computation of autosome or other chromosome ratios, this count should not be subject to weighting according to fragment size.

### Ratio Calculation

Once the total number of hits have been assigned to a given chromosome in accordance with the fragment alignment analysis herein defined, the hits are then typically normalised to a common number. The ratio of each hits for a target region of a target chromosome compared with hits on one or more reference chromosomes is then calculated in accordance with simple mathematics.

In addition to normalization to a common number as referred to hereinbefore, it is typically useful to be able to estimate the fraction of the maternal plasma DNA that is fetal in origin;

this will confirm that there is sufficient fetal DNA in a sample of maternal plasma DNA for detecting a fetal chromosomal abnormality. For example, in one embodiment, the method of the invention additionally comprises the step of normalizing or adjusting the number of matched hits based on the amount of fetal DNA within the sample.

### Statistical Significance

In order to place the diagnostic test of the invention on a statistical basis, the method of the invention additionally comprises the step of calculating statistical significance of the ratio of each hits for a target region of a target chromosome compared with hits on other chromosomes. In one embodiment, the statistical significance test comprises calculation of the z-score in accordance with conventional statistical analysis of the reduced counting data. However, it will be appreciated that other statistical methods may be applied by skilled workers in the field.

Where the distribution of the errors in the counts ratio “target chromosome/one or more reference chromosomes” is assumed to be approximately normal, the z-score indicates how many standard deviations an element is from the mean.

A z-score can be calculated from the following formula:

z=(X−μ)/σ

wherein z is the z-score, X is the value of the element, p is the population mean, and a is the standard deviation of the population values. When testing for the presence of Trisomy 21 according to the present invention, a z-score value of 2.0 or more for the count ratio indicates a probability of approx 98% that the count ratio value indicates a Trisomy 21 pregnancy.

In one embodiment, step (e) comprises calculation of a likelihood ratio which is indicative of a fetal chromosomal abnormality for a target chromosome and is typically based upon a number of factors, such as the fetal fraction, the above mentioned z-score etc. Full details of how a likelihood ratio may be calculated are described in WO 2014/033455.

### Methods of Predicting Gender

The presence of Chromosome Y DNA, which is inherited from the paternal parent of the fetus, is a diagnostic marker of a male fetus. A further aspect of the present invention is the detection of the gender of the fetus as indicated by the presence of Chromosome Y sequences.

Where the fetus is female the use of the Y chromosomal component is precluded, however in place of the paternally-inherited Y-chromosome, it is possible to detect gene alleles that are paternally-derived. Among these are fetal SNPs (single nucleotide polymorphisms), which are evident as alleles present as a minor component of the DNA sequences in maternal plasma DNA (Dhallan et al., Lancet 369, 474-481). Where a fraction of the fetal genome only is sequenced, as in the present invention, the number of such alleles inherited from the fetus' father, and detected as variants differing from the relatively more abundant maternal alleles, is a function of the fraction of the plasma DNA that is fetal. This provides an alternative, gender-independent, method for estimating the fraction of maternal plasma DNA that is fetal in origin.

According to a second aspect of the invention there is provided a method of predicting the gender of a fetus within a pregnant female subject, the method comprising the steps of:
(a) isolating nucleic acids from within a biological sample obtained from a pregnant female subject;
(b1) selecting a nucleic acid fragment size value of between 120 bp and 135 bp for optimal fetal fraction;
(b2) isolating nucleic acid fragments having a size within 20 bp of the fragment size value selected in step (b1);
(c) determining a first number of said fragments which align to a sex chromosome and determining a second number of said fragments which align to one or more reference chromosomes;
(d) calculating a ratio or difference between the first and second numbers;
(e) determining the gender of said fetus based on whether an excess or equivalence of fragments align to an X chromosome compared to said reference chromosome or whether a Y chromosome is present or absent.

According to a further aspect of the disclosure which may be mentioned there is provided a method of predicting the gender of a fetus within a pregnant female subject, the method comprising the steps of:
(a) isolating nucleic acids from within a biological sample obtained from a pregnant female subject;
(b) isolating nucleic acid fragments having a size from approximately 80 bp to approximately 150 bp;
(c) determining a first number of said fragments which align to a sex chromosome and determining a second number of said fragments which align to one or more reference chromosomes;
(d) calculating a ratio or difference between the first and second numbers;
(e) determining the gender of said fetus based on whether an excess or equivalence of fragments align to an X chromosome compared to said reference chromosome or whether a Y chromosome is present or absent.

In one embodiment, the method additionally comprises the steps:
(i) size-weighting each fragment which aligns to a sex chromosome by calculating the probability (w) of each fragment size (s) being fetal in origin;
(ii) size-weighting each fragment which aligns to one or more reference chromosomes by calculating the probability (w) of each fragment size (s) being fetal in origin;
(iii) calculating a total target weighted count (Nc_{target}) by summing the values obtained in step (i);
(iv) calculating a total reference weighted count (Nc) by summing the values obtained in step (ii);
(v calculating a ratio or difference between the Nc_{target} and Nc values obtained in steps (iii) and (iv); and
(vi) determining the gender of said fetus based on whether an excess or equivalence of fragments align to an X chromosome compared to said one or more reference chromosomes or whether a Y chromosome is present or absent.

References herein to sex chromosome, i.e. allosome, include either the X or Y chromosome.

In embodiment, the reference chromosome is selected from an autosome (i.e. non-sex chromosome).

It will be appreciated that an excess of fragments aligning to an X chromosome compared to said reference chromosome is indicative of a female gender prediction (i.e. XX).

It will be appreciated that an equivalence of fragments aligning to an X chromosome compared to said reference chromosome is indicative of a male gender prediction (i.e. XY).

It will be appreciated that the presence of fragments aligning to a Y chromosome is indicative of a male gender prediction (i.e. XY).

It will be appreciated that the absence of fragments aligning to a Y chromosome is indicative of a female gender prediction (i.e. XX).

It will be appreciated that each of the embodiments which apply to the first aspect of the invention apply equally to the gender prediction method of the second aspect of the invention.

### Kits

According to a further aspect of the invention, there is provided a kit for performing any of the methods defined herein which comprises instructions for use of the kit in accordance with any of the methods defined herein.

In one embodiment, the kit additionally comprises one or more reagents and/or one or more consumables as defined herein.

According to a further aspect of the invention, there is provided the use of a kit as defined herein in a method of detecting a fetal chromosomal abnormality within a pregnant female subject or a method of predicting the gender of a fetus within a pregnant female subject.

The following studies illustrate the invention.

### Materials and Methods

It will be appreciated that the methods described herein may be conducted using the IONA® test which may be seen at http://www.premaitha.com/the-iona-test. However, the following detailed protocol provides guidance regarding how the method of the invention may be carried out.

### Materials In addition to the reagents provided in the IONA® Library Preparation Kit, the following are required for DNA library preparation using the manual protocol:

Freezer
Refrigerator
Pipettes
Pipette tips
50 mL tubes
Vortex
Plate centrifuge
Microcentrifuge(s), suitable for use with 0.2 mL and 1.5 mL tubes
Microcentrifuge tubes—0.2 mL and 1.5 mL, with low DNA binding capacity
Magnetic rack/plate
Thermal cycler, suitable for use with 96-well PCR plates
Nuclease-free water
Molecular biology grade Ethanol
Bioanalyser instrument, e.g. Perkin Elmer LabChip® GX, Agilent 2100 Bioanalyser0
Bioanalyser0 reagents, e.g. HT DNA 1K/12K/High Sensitivity LabChip® and HT DNA Hi Sensitivity Reagent Kit (Cat. Nos. 760517 & CLS760672; Perkin Elmer) Agilent High Sensitivity DNA Analysis Kit (Cat. No. 5067-4626; Agilent Technologies)
Note: If a 96-well plate magnet is used for the manual library preparation protocol, consumables provided in the IONA® Plastics Consumables Kit can be used.

### DNA Extraction

The IONA® test utilises cell-free DNA (cfDNA) derived from the plasma fraction of whole blood as the input sample for analysis. When performing a manual DNA extraction protocol for use in the IONA® test workflow, a DNA extraction kit validated for use in extracting cfDNA from plasma must be used.

Sample processing should be performed according to the instructions provided by the DNA extraction kit manufacturer, or to established procedures known to those skilled in the art.

### DNA Library Preparation

The manual protocol for DNA library preparation in the IONA® test utilises the reagents provided in the IONA® Library Preparation Kit. Batching of samples is recommended when using the manual protocol for the IONA® Library Preparation Kit to avoid reduced sample throughput, in comparison with the automated protocol.
1. Remove the DNA from the freezer (if required) and thaw for 30 minutes at ambient temperature (15 to 25° C.).
2. Remove the IONA® Library Preparation Kit Plate 1 from the freezer and thaw for 30 minutes at ambient temperature (15 to 25° C.).
3. Remove the IONA® Library Preparation Kit Plate 2 from the refrigerator and equilibrate to ambient temperature (15 to 25° C.) for a minimum of 30 minutes.
4. Pulse spin the IONA® Library Preparation Kit Plates 1 and 2 in a plate centrifuge for 5 seconds to collect reagents at the bottom of the plate.
   Note: The IONA® Library Preparation Kit Plate 2 remains on the benchtop at ambient temperature until required at subsequent steps.
5. Reagent layouts for IONA® Library Preparation Kit Plates 1 and 2 are described below in Tables 1 and 2.

TABLE 1
Plate Layout for the IONA ® Test Plate 1
IONA ® Library Preparation Plate 1

| | 1 | 2 | 3 | 4 | 5 | 6 | 7 | 8 | 9 | 10 | 11 | 12 |
|---|---|---|---|---|---|---|---|---|---|---|---|---|
| | ER Buff.:1 | ER Enz: | ALB I: | ALE I: | ALE II: | Adapt.: | Adapt.: | Adapt.: | Adapt.: | Primer: | PCR Mix: | PCR Mix: |
| | 102 | 1101 | 1103 | 1104 | 1105 | 1201-1204 | 1205-1208 | 1209-1212 | 1213-1216 | 1106 | 1107 | 1107 |
| A | 27 μL | 117 μL* | 45 μL | 27 μL | 39 μL | Not used | 1205: 21 μL | Not used | 1213: 21 μL | 45 μL | 117 μL | 117 μL |
| B | 27 μL | Not used | 45 μL | 27 μL | Not used | 1201: 21 μL | Not used | 1209: 21 μL | Not used | 45 μL | 117 μL | 117 μL |
| C | 27 μL | Not used | 45 μL | 27 μL | Not used | Not used | 1206: 21 μL | Not used | 1214: 21 μL | Not used | 117 μL | 117 μL |
| D | 27 μL | Not used | 45 μL | 27 μL | Not used | 1202: 21 μL | Not used | 1210: 21 μL | Not used | Not used | 117 μL | 117 μL |
| E | 27 μL | Not used | 45 μL | 27 μL | Not used | Not used | 1207: 21 μL | Not used | 1215: 21 μL | Not used | 117 μL | 117 μL |
| F | 27 μL | Not used | 45 μL | 27 μL | Not used | 1203: 21 μL | Not used | 1211: 21 μL | Not used | Not used | 117 μL | 117 μL |
| G | 27 μL | Not used | 45 μL | 27 μL | Not used | Not used | 1208: 21 μL | Not used | 1216: 21 μL | Not used | 117 μL | 117 μL |
| H | 27 μL | Not used | 45 μL | 27 μL | Not used | 1204: 21 μL | Not used | 1212: 21 μL | Not used | Not used | 117 μL | 117 μL |

*The IONA Library Preparation Kit HT contains 126 μL.

TABLE 2
Plate Layout for the IONA ® test Plate 2

| | 1 | 2 | 3 | 4 | 5 | 6 | 7 | 8 | 9 | 10 | 11 | 12 |
|---|---|---|---|---|---|---|---|---|---|---|---|---|
| | Library Preparation Reagent Plate 2 | | | | | | | | | | | |
| A | 117 μL | 117 μL | 117 μL | 117 μL | 117 μL | 117 μL | 117 μL | 117 μL | 117 μL | 117 μL | 117 μL | 117 μL |
| B | 117 μL | 117 μL | 117 μL | 117 μL | 117 μL | 117 μL | 117 μL | 117 μL | 117 μL | 117 μL | 117 μL | 117 μL |
| C | 117 μL | 117 μL | 117 μL | 117 μL | 117 μL | 117 μL | 117 μL | 117 μL | 117 μL | 117 μL | 117 μL | 117 μL |
| D | 117 μL | 117 μL | 117 μL | 117 μL | 117 μL | 117 μL | 117 μL | 117 μL | 117 μL | 117 μL | 117 μL | 117 μL |
| E | 117 μL | 117 μL | 117 μL | 117 μL | 117 μL | 117 μL | 117 μL | 117 μL | 117 μL | 117 μL | 117 μL | 117 μL |
| F | 117 μL | 117 μL | 117 μL | 117 μL | 117 μL | 117 μL | 117 μL | 117 μL | 117 μL | 117 μL | 117 μL | 117 μL |
| G | 117 μL | 117 μL | 117 μL | 117 μL | 117 μL | 117 μL | 117 μL | 117 μL | 117 μL | 117 μL | 117 μL | 117 μL |
| H | 117 μL | 117 μL | 117 μL | 117 μL | 117 μL | 117 μL | 117 μL | 117 μL | 117 μL | 117 μL | 117 μL | 117 μL |

6. Prepare 51 μL of each DNA sample for testing in an individual microcentrifuge tube or in a 96-well plate suitable for the verified thermal cycler.
7. Mix the End Repair Buffer I in column 1 and the End Repair Enzyme I in position A2 of the IONA® Library Preparation Kit Plate 1 (Table 2) by pipetting up and down 10 times. Note: It is important to perform this for any reagent well which is to be used.
8. Add the following volumes of each reagent of the End Repair reaction to each sample in order to prepare the reaction:

TABLE 3
End Repair Reaction Volumes

| | Plate 1 Column | | |
|---|---|---|---|
| Reagent | for Reagent | Volume | |
| Sample | — | 51 | μL |
| End Repair Buffer I | 1 | 6 | μL |
| End Repair Enzyme I | A2 | 3 | μL |
| Total | | 60 | μL |

Note: A master mix for the End Repair reagents can be prepared for this step if multiple DNA samples are being tested. An overage of at least one reaction is recommended. The number of samples that can be tested using the IONA® Library Preparation Kit may be reduced if using this method.
9. For each sample, pipette the End Repair reaction up and down 10 times to mix and pulse spin for 5 seconds using an appropriate benchtop centrifuge.
10. Store the IONA® Library Preparation Kit Plate 1 in a refrigerator until required for the Adaptor Ligation reaction.
11. Transfer each sample tube (or 96-well reaction plate) to a thermal cycler to perform the End Repair reaction. Set the thermal cycler to the following cycling conditions and run the End Repair reaction:

TABLE 4
End Repair Temperature Cycling

| Temperature | Time | |
|---|---|---|
| 25° C. | 20 | minutes |
| 70° C. | 10 | seconds |

| | |
|---|---|
| 4° C. | ∞ |

Note:
Ensure that the volume for the reaction is set to 60 μL

12. On completion of the End Repair reaction, transfer the samples from the thermal cycler to the benchtop for preparation of the Adaptor Ligation reaction.
13. Remove the IONAO Library Preparation Kit Plate 1 from the refrigerator. Pipette the Adaptor Ligation Buffer I (ALB I) in column 3, the Adaptor Ligation Enzyme I (ALE I) in column 4 and the Adaptor Ligation Enzyme II (ALE II) in position A5 of the IONAO Library Preparation Kit Plate 1 (Table 2) up and down 10 times to mix.
   Note: Perform this for any reagent well which is going to be used.
14. Add the following volumes of each reagent of the Adaptor Ligation reaction directly to each sample from the End Repair reaction:

TABLE 5
End Repair Temperature Cycling

| | Plate 1 Column | | |
|---|---|---|---|
| Reagent | for Reagent | Volume | |
| Sample (from End Repair) | — | 60 | μL |
| Nuclease-free water | — | 15 | μL |
| Adaptor Ligation Buffer I | 3 | 10 | μL |
| Adaptor Ligation Enzyme I | 4 | 6 | μL |
| Adaptor Ligation Enzyme II | A5 | 1 | μL |
| | | 92 | μL |

Note: A master mix for the End Repair reagents can be prepared for this step if multiple DNA samples are being tested. An overage of at least one reaction is recommended. The number of samples that can be tested using the IONA® Library Preparation Kit may be reduced if using this method.
15. Pipette the barcoded adaptors in columns 6 to 9 of the IONA® Library Preparation Kit Plate 1 (Table 2) up and down 10 times to mix.
   Note: Perform this for the appropriate number of samples being prepared. Use one barcoded adaptor per sample.
16. Add 8 μL of the barcoded adaptor assigned to the appropriate sample to the Adaptor Ligation reaction for the sample. The Adaptor Ligation reaction volume is 100 μL.
   Note: The barcoded adaptor number (Table 2; Columns 6-9) used for each sample must be recorded as each sample will be analysed in subsequent steps according to its individual barcode.
17. For each sample, pipette the Adaptor Ligation reaction up and down 10 times to mix and pulse spin for 5 seconds using an appropriate benchtop centrifuge.
18. Store the IONA® Library Preparation Kit Plate 1 in a refrigerator until required for the Library PCR reaction.
19. Transfer each sample to the verified thermal cycler to perform the Adaptor Ligation reaction. Set the thermal cycler to the following cycling conditions and run the Adaptor Ligation reaction:

TABLE 6
Adaptor Ligation Cycling

| | | |
|---|---|---|
| Hold | 25° C. | 15 min |
| Hold | 65° C. | 5 min |
| Hold | 4° C. | ∞ |

Note: Ensure that the volume for the reaction is set to 100 μL.
20. On completion of the Adaptor Ligation reaction, transfer the samples from the thermal cycler to the benchtop for clean-up of the Adaptor Ligation reaction.
21. (If required) Transfer each sample to an appropriate vessel for use with the magnetic plate/rack available for sample clean-up.
22. Prepare a solution of 80% ethanol (50 mL total volume) by mixing 40 mL of 100% ethanol with 10 mL nuclease free water.
23. Pipette the beads in the IONA® Library Preparation Kit Plate 2 (Table 3) stored at ambient temperature (15 to 25° C.) up and down 25 times to mix.
   Note: Perform this for any well that the beads will be taken from. Adequate mixing of the beads is important.
24. Add 100 μL of the beads directly to each sample from the Adaptor Ligation reaction and pipette up and down 10 times to mix.
25. Incubate each sample for 5 minutes at ambient temperature (15 to 25° C.).
26. Pulse spin the samples in an appropriate benchtop centrifuge for 5 seconds.
27. Transfer the samples (in 1.5 mL tubes or 96-well plate) to the magnetic plate/rack for 2 minutes.
28. While keeping the samples on the magnetic plate/rack, remove and discard the supernatant. Be careful not to disturb the pellet.
29. Add 80% ethanol to each sample. Ensure that the entire bead pellet is immersed. Note: If using a 96-well plate, 200 μL of 80% ethanol is recommended. If using 1.5 mL tubes, 500 μL of 80% ethanol is recommended.
30. Incubate each sample for 30 seconds in 80% ethanol.
31. Keep the samples on the magnetic plate/rack. Remove and discard the supernatant. Be careful not to disturb the pellet.
32. Repeat steps 29 to 31.
33. Air dry each sample on the magnetic plate/rack for 5 minutes at ambient temperature (15 to 25° C.).
   Note: If using 1.5 mL tubes, ensure the tube caps are open.
34. Remove each sample off the magnetic plate/rack and re-suspend the bead pellet in 43 μL of nuclease-free water.
   Note: Ensure all beads are in suspension. Pipette along the side of the plate well/tube on which the beads were held against the magnet in order to recover the entire bead pellet.
35. Incubate each sample for 3 minutes at ambient temperature (15 to 25° C.).
36. Pulse spin the samples in an appropriate benchtop centrifuge for 5 seconds.
37. Transfer the samples to the magnetic plate/rack for 2 minutes.
38. Transfer 40 μL of the supernatant to a fresh plate well/tube for library PCR.
39. Remove the IONAO Library Preparation Kit Plate 1 from the refrigerator. Pipette the PCR Primer Mix I in positions 10A/B and the PCR Master Mix I in columns 11 and 12 of the IONA® Library Preparation Kit Plate 1
40. Pipette up and down 10 times to mix
   Note: Perform this for any reagent well which is to be used.
41. Add the following volumes of each reagent of the Library PCR reaction directly to each sample from the clean-up of the Adaptor Ligation reaction:

TABLE 7

| | Plate 1 Column | | |
|---|---|---|---|
| Reagent | for Reagent | Volume | |
| Sample | — | 40 | μL |
| Nuclease-free water | — | 8 | μL |
| PCR Primer Mix I | 10 | 2 | μL |
| PCR Master Mix I | 11 & 12 | 50 | μL |
| | | 100 | μL |

Note: A master mix for the Library PCR reagents can be prepared for this step if multiple DNA samples are being tested. An overage of at least one reaction is recommended. The number of samples that can be tested using the IONA® Library Preparation Kit may be reduced if using this method.
42. For each sample, pipette the Library PCR reaction up and down 10 times to mix and pulse spin for 5 seconds using an appropriate benchtop centrifuge.
   Note: Return the IONA® Library Preparation Kit Plate 1 to the freezer for storage.
43. Transfer each sample to a thermal cycler to perform the Library PCR reaction Set the thermal cycler to the following cycling conditions and run the Library PCR reaction :

TABLE 8
Library PCR

| | | | |
|---|---|---|---|
| Denature | 98° C. | 30 | s |
| Cycling (x12) | 98° C. | 10 | s |
| | 58° C. | 30 | s |
| | 72° C. | 30 | s |
| Hold | 72° C. | 5 | min |

| | | |
|---|---|---|
| Hold | 4° C. | ∞ |

Note: Ensure that the volume for the reaction is set to 100 μL.
44. On completion of the Library PCR reaction, transfer the samples from the thermal cycler to the benchtop for library quantification.
   Note: PCR amplified libraries can be stored in the freezer (−15 to −25° C.) and the workflow completed within 20 working days. Return the IONA® Library Preparation Kit Plate 2 to the refrigerator for storage until required.
Note: If commencing the workflow from this point after storage of PCR amplified libraries in the freezer (−15 to −25° C.), thaw libraries for 30 minutes prior to subsequent steps. Ensure IONA® Library Preparation Kit Plate 2 is removed from storage in the refrigerator for 30 minutes for subsequent steps.
45. Perform quantification for each of the sample libraries using a DNA analyser platform (e.g. Perkin Elmer LabChip® GX, Agilent 2100 Bioanalyser®) according to the manufacturer's instructions.
   Note: PCR amplified libraries may be too concentrated to be run undiluted on certain DNA analyser platforms. It is recommended that a ⅕ dilution is prepared of each library to be quantified.
   Note: The concentration of each library must be recorded in molarity for subsequent normalisation and multiplexing, prior to sequencing. Correct the concentrations for dilution factors as required.
   Note: Ensure that the concentration of any library being quantified is within the limits of detection of the DNA analyser platform being used.
46. Perform the normalisation and multiplexing of samples prior to size selection using the following steps. Up to 8 samples can be multiplexed for sequencing in a single run. Ensure sample libraries with the same barcoded adaptor number are NOT added in the same multiplexed pool.
47. Select up to 8 samples and their corresponding concentrations (molarity; nM) from quantification. Identify the sample with the lowest concentration—this value is the TARGET concentration all samples will be normalised to.
48. Use the following calculation for each sample to be pooled to determine the volumes required for the multiplexing of libraries:

### Sample Library Volume

TARGET concentration (nM)×20 μL Sample Library concentration (nM)

### Water Volume

20 μL—Sample Library volume
e.g. Sample 5 =Lowest Concentration =TARGET Concentration Volumes underlined in the example indicate final volumes to be added.

TABLE 9

| | Concentra- | | |
|---|---|---|---|
| Sample | tion (nM) | Sample Vol (μL) | Water Vol (μL) |
| Sample 1 | 1.2 | 1/1.2 × 20 = 16.7 | 20 − 16.7 = 3.3 |
| Sample 2 | 1.4 | 1/1.4 × 20 = 14.3 | 20 − 14.3 = 5.7 |
| Sample 3 | 1.6 | 1/1.6 × 20 = 12.5 | 20 − 12.5 = 7.5 |
| Sample 4 | 1.8 | 1/1.8 × 20 = 11.1 | 20 − 11.1 = 8.9 |
| Sample 5 (Lowest) | 1 | 1/1 × 20 = 20 | 20 − 20 = 0 |
| Sample 6 | 2 | 1/2 × 20 = 10 | 20 − 10 = 10 |
| Sample 7 | 2.2 | 1/2.2 × 20 = 9.1 | 20 − 9.1 = 10.9 |
| Sample 8 | 2.4 | 1/2.4 × 20 = 8.3 | 20 − 8.3 = 11.7 |
| Total Volume (μL)= | 160= | 102 + | 58 |

49. In a 1.5 mL tube, combine the water and sample library volumes calculated for the normalisation of all samples to be multiplexed.
   Note: A minimum volume of 100 μL of the pooled samples is required for subsequent steps. If fewer than 5 samples are being multiplexed, the 20 μL volume shown in the calculation above can be increased as necessary.

### Enrichment Using Size Selection (using Ranger Technology™)

### Setup

Select the appropriate Dual-Dye Loading Buffer (dependent on loading volume) and agarose cassettes for the samples.
Combine the Dual-Dye Loading Buffer and sample in a single 0.2 ml microcentrifuge tube.
   Size selection (28.5 μl volume): 3.5 μl loading buffer+25 μl sample
   Size selection (50 μl volume): 6 μl loading buffer+44 μl sample
Pipette mix and spin down tube.
Prime and calibrate the cassette using;
   28.5 μl volume cassette =15 μl (loading well)+75 μl (extraction well) of [TBE]
   50 μl volume cassette =25 μl (loading well)+75 μl (extraction well) of [TBE]
Place cassette onto deck
Start software
Once prompted, skip cleaning step
−4×50 μl extraction prompts to extract from extraction well regardless of loading volume.

### Software

Open the Ranger Software
Select the appropriate deck layout (File>New>Run>)
Define the contents of the source plate (click on the source plate image)
Define the agarose-type of each cassette (fully lasso each cassette, then right-click to select the gel type)
Sample type manager; 100-300 bp marker, Specify target base pair range (205-227 bp), 100% speed.
Define the agarose percentage type of the cassette e.g. 2%, 3%
Perform a visual inspection of the deck and close the instrument door.
Click the software ‘Start’ button to begin the run.
Follow the on-screen prompts throughout the duration of the run and complete the appropriate washes and extractions
Ranger Technology™ run completes, remove sample

Transfer 200 μL of the size selected sample libraries from the Ranger Technology™. Split into 2×100 ul reactions, each mixed with 700 ul of beads for clean up of sample.

Pipette the beads in the IONA® Library Preparation Kit Plate 2 stored at ambient temperature (15 to 25° C.) up and down 25 times to mix.
Note: Perform this for any reagent well which is to be used.

TABLE 10
Plate Layout for the IONA ® test Plate 2
Table 2: Plate Layout for the IONA ® test Plate 2

| | 1 | 2 | 3 | 4 | 5 | 6 | 7 | 8 | 9 | 10 | 11 | 12 |
|---|---|---|---|---|---|---|---|---|---|---|---|---|
| | Library Preparation Reagent Plate 2 | | | | | | | | | | | |
| A | 117 μL | 117 μL | 117 μL | 117 μL | 117 μL | 117 μL | 117 μL | 117 μL | 117 μL | 117 μL | 117 μL | 117 μL |
| B | 117 μL | 117 μL | 117 μL | 117 μL | 117 μL | 117 μL | 117 μL | 117 μL | 117 μL | 117 μL | 117 μL | 117 μL |
| C | 117 μL | 117 μL | 117 μL | 117 μL | 117 μL | 117 μL | 117 μL | 117 μL | 117 μL | 117 μL | 117 μL | 117 μL |
| D | 117 μL | 117 μL | 117 μL | 117 μL | 117 μL | 117 μL | 117 μL | 117 μL | 117 μL | 117 μL | 117 μL | 117 μL |
| E | 117 μL | 117 μL | 117 μL | 117 μL | 117 μL | 117 μL | 117 μL | 117 μL | 117 μL | 117 μL | 117 μL | 117 μL |
| F | 117 μL | 117 μL | 117 μL | 117 μL | 117 μL | 117 μL | 117 μL | 117 μL | 117 μL | 117 μL | 117 μL | 117 μL |
| G | 117 μL | 117 μL | 117 μL | 117 μL | 117 μL | 117 μL | 117 μL | 117 μL | 117 μL | 117 μL | 117 μL | 117 μL |

Incubate the sample for 5 minutes at ambient temperature (15 to 25° C.).

Pulse spin the sample in an appropriate benchtop centrifuge for 5 seconds. Transfer the sample to the magnetic plate/rack for 2 minutes.

While keeping the sample on the magnetic plate/rack. Remove and discard the supernatant. Be careful not to disturb the bead pellet.

Add 80% ethanol to the sample. Ensure the entire bead pellet is immersed.

Note: If using a 96-well plate, 200 μL of 80% ethanol is recommended. If using 1.5 mL tubes, 500 μL of 80% ethanol is recommended.

Incubate the sample for 30 seconds in 80% ethanol.

While keeping the sample on the magnetic plate/rack. Remove and discard the supernatant. Be careful not to disturb the bead pellet.

Repeat steps 56 to 58.

Air dry the sample on the magnetic plate/rack for 5 minutes at ambient temperature (15 to 25° C.).

Note: If using a 1.5 mL tube, ensure the tube cap is open.

Remove the sample off the magnetic plate/rack and re-suspend the bead pellet in 18 μL of nuclease-free water.

Note: Ensure all beads are in suspension. Pipette along the side of the plate well/tube on which the beads were held against the magnet in order to recover the entire bead pellet.

Incubate the sample for 3 minutes at ambient temperature (15 to 25° C.).

Pulse spin the sample in an appropriate benchtop centrifuge for 5 seconds.

Transfer the sample to the magnetic plate/rack for 2 minutes.

Transfer 2×15 μL of the supernatant to a fresh plate well/tube.

Perform quantification of the size-selected, multiplexed sample using a DNA analyser platform (e.g. Perkin Elmer LabChip® GX, Agilent 2100 Bioanalyser®) according to the manufacturer's instructions.

Note: The size-selected, multiplexed sample may be run undiluted on the DNA analyser platform. Ensure that the concentration of the sample is within the limits of detection of the DNA analyser platform being used. The sample may be diluted and re-quantified as necessary.

Note: The concentration of each library must be recorded in molarity for subsequent dilutions for sequencing. Correct the concentrations for dilution factors as required.

Use the concentration determined for the size-selected, multiplex sample pool from the DNA analyser to perform the dilution to the required input concentration for the next generation sequencing platform to be used.

The IONA® test has been validated using the Ion Chef™ instrument and the Ion Proton™ next generation sequencing platform (Thermo Fisher), using an input concentration of 40 pM (50 pM if using Ion PI V2 chips) for the final size-selected, multiplex sample pool. Use the following calculation to determine the volumes required for the sample dilution:

### Sample Library Volume

Target concentration =40 pM (or 50 pM)×(120 μL)×μL required of sample library

### Sample Library Concentration

### Run Control 1/25 Preparation

2 μL Stock Run Control+48 μL water=50 μL of 1/25 Run Control

### Sample preparation for sequencing:

×μL required of sample library+22.2 μL 1/25 Run Control
Bring up to a final volume with water to 120 μL total volume
Note: It is recommended that this dilution is performed immediately before the reaction set-up for the next generation sequencing platform to be used.
Note: Avoid freezing the diluted multiplexed sample, as possible effects from freeze/thaw cycling are more detrimental for DNA at low concentrations. Storage of the diluted sample in the refrigerator for up to 24 hours prior to the reaction set-up for sequencing may be performed.

### Next Generation Sequencing Reaction Set-up

The next generation sequencing reaction can be performed using a semi-automated or fully automated protocol.

The IONA® test has been validated using the Ion Chef™ instrument and the Ion Proton™ next generation sequencing platform (Thermo Fisher). The workflow for this automated DNA library protocol is described below.

Note: For alternative protocols, prepare the sample in accordance with the manufacturer's protocol.

The following consumables are required for next generation sequencing using the Ion Chef™ and the Ion Proton™ instruments:
Ion PI Chip Kit V3 of Ion PI V2 BC (Cat. No. A26771 or 4484270; Thermo Fisher)
Ion PI Hi-Q Chef kit or Ion PI IC 200 Kit (Cat. No.A27198 or 4488377; Thermo Fisher)
Sodium hydroxide solution (e.g. Cat. No. 10488790; Fisher Scientific)

Isopropanol, molecular biology grade if using Ion PI V2 BC chips (e.g. Cat. No. 11388461; Fisher Scientific)

Next generation sequencing runs, using the automated protocol for the Ion Chef™ and Ion Proton™ platforms, are performed in accordance with the protocols outlined in the Ion PI™ Hi-Q Chef of IC 200 Kit User Guide.

If not already prepared, dilute the size-selected, multiplexed samples to be tested to the required input concentration described in step 88 of the manual library preparation protocol.

Plan the Ion Chef TM/Ion Proton™ runs to be performed in accordance with the manufacturer's instructions.

Note: Scanning of the barcodes on the sequencing chip during run set-up is performed to pair the correct multiplexed library sample pool with the correct chip. The assignment of the appropriate adaptor barcode with Sample IDs is performed by the IONA® Analysis software.

Note: If you are using an alternative next generation sequencing protocol or platform, ensure the correct sample IDs are assigned to the correct adaptor barcodes.

Note: Two sequencing reactions and runs are prepared for each set-up of the Ion Chef™ and Ion Proton™instruments. The workflow occurs over two days. Completion time for the Ion Chef™ reaction can be selected in advance, to allow a reaction to be performed overnight.

On completion of the Ion Proton™ sequencing run, the data is transferred directly to be analysed by the IONA® Software to determine the likelihood status for the trisomies investigated for all samples tested.

Note: Data transfer and analysis following sequencing can take up to 6 hours. Do not turn off the Ion Proton™ instrument or the IONA® software workstation PC during this time.

### Data Analysis—IONA Software

In summary, the following steps are conducted:
Samples de-multiplexed;
Reads aligned & binned into chromosomes;
Reads GC corrected; and
Chromosome ratios calculated, FF% estimates calculated.

Execution of the main bioinformatics pipeline proceeds as follows through the use of the IONA® Software: For each sequencing run of eight samples, multiplexed sequence reads are retrieved from the sequencing platform in the form of an unmapped BAM file. The multiplexed assembly of reads is initially subject to a barcode classification step, in which barcoded 5′ adapters are identified and matched against a predefined set, in order to split the multiplex into reads against individual samples for further processing.

Following an early filtering step to remove a small number of very short reads, fragments are mapped to the ‘hg19’ human genome reference using a gap-tolerant read alignment module. Post-filtering of alignment results is then carried out to remove duplicate reads arising in PCR stages of the test workflow, determined as those whose 5′ end map to the reference at the same position as any other read.

Fragments determined to have aligned uniquely in the genome reference are then binned by autosome, with the resulting counts subject to a calibration step to correct sequencing coverage bias correlated to GC content; this is achieved by first characterising the level of over- or under-representation of fragments according to their average GC content when binned across the genome reference, and then inverting and applying as a corrective weighting to fragment counts per chromosome.

Finally, the resulting fragment count data are used as input to a set of mixture models that incorporate distributions of expected values under both trisomy-affected and unaffected hypotheses for trisomy 13, 18 and 21 tests. Each model generates a test likelihood ratio that is then used, together with maternal age-derived prior probabilities of trisomy, to quantify the probability of each trisomy taking into account both age and the corresponding DNA test result.

The IONA® Software also performs internal validity checks. Workflow data quality checks take place, which make use of sequencing and alignment metrics to ensure sequence data are of sufficient quality for further analysis to take place. Additionally, following the generation of per-autosome fragment counts, the run validity check takes place. This step first isolates fragments derived from sequencing an In-Run Control designed to simulate a

Trisomy 21-positive sample with approximately 10% fetal fraction, and then compares the proportion of counts from these fragments which aligned against chromosome 21 using a reference range previously set in the software configuration. If the proportion meets the reference criteria, the run validity check passes.

Separate validity checks also take place for each sample. These ensure that the aligned fragment count is sufficient for the likelihood mixture model to be used, and that the fraction of cfDNA in the sample that was of fetal origin is sufficient for a result to be reported. For this last check, fetal fraction is first independently quantified using a combination of measurement of X chromosome representation (where possible, i.e. in male fetal cases) and a method which assesses the relative amount of material in a fetally-enriched size region where X chromosome representation is not informative for fetal fraction.

### 1. Enrichment Results

The results described in this document demonstrate a substantial enrichment of fetal DNA relative to the maternal background DNA content.

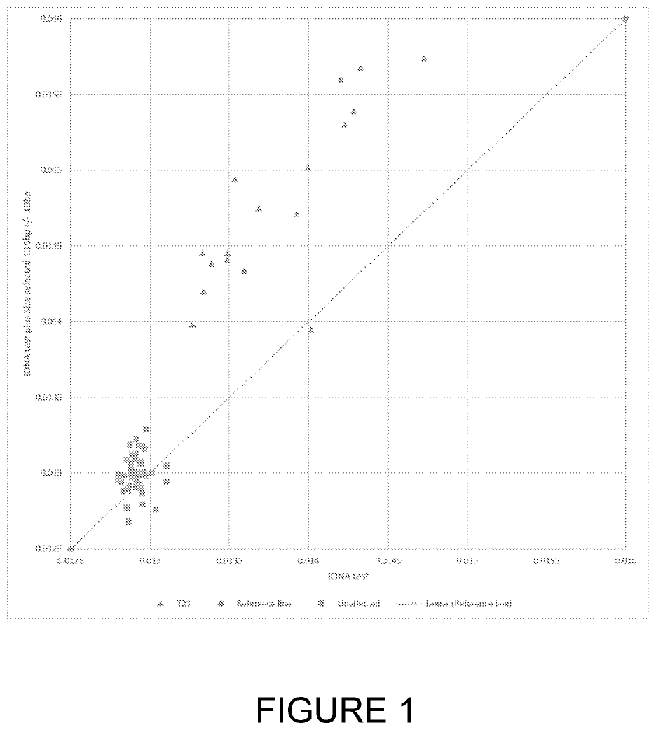

FIG. 1 illustrates the effect of enrichment using the fragment size enrichment method on chromosome 21 ratios. The ratios for unaffected (euploid) samples (squares) clusters around the expected values; i.e., no change relative to the reference result, whereas the chromosome 21 ratios for Trisomy 21 samples (triangles) are significantly increased relative to the reference result. The enrichment method has significantly increased the difference in

Chromosome 21 ratio between the euploid sample with the highest ratio and the T21 sample with the lowest ratio. This vastly improves the ability to distinguish between euploid and trisomy samples. The data generated in FIG. 1 demonstrates enrichment of chromosome 21 DNA which can only occur through enrichment of the fetal component. In this dataset, 20 of 21 samples are enriched in this manner, with one T21 sample that is not enriched. The enriched data was generated using 32 sample multiplexing during the sequencing steps, whereas the reference data is 8-16 sample multiplexing. It would be expected that the 32plex data should show poorer discrimination between T21 and unaffected samples (due to the reduced amount of data per sample), however the results demonstrate that it is improved due to enrichment.

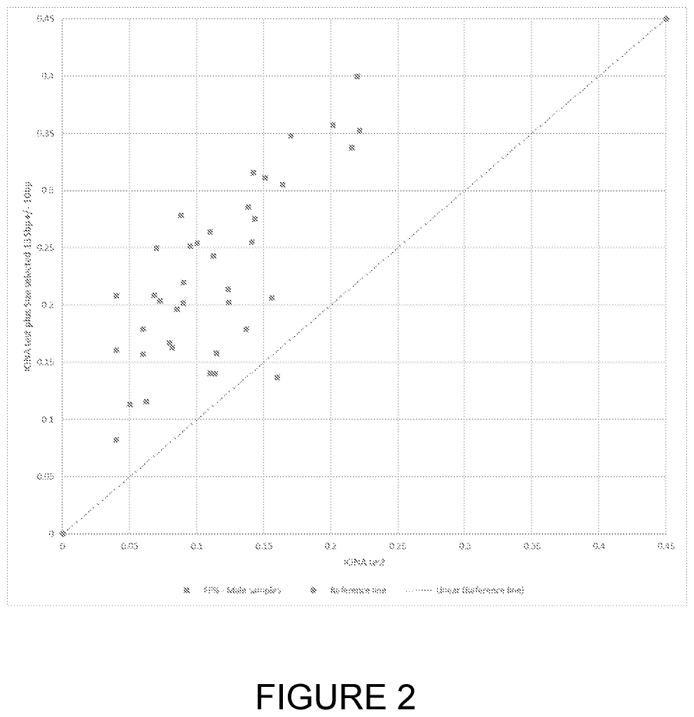

FIG. 2 illustrates the effect of enrichment using the fragment size enrichment method on fetal fraction estimates in male samples. The data demonstrates that the proportion of DNA originating from the fetus is substantially enriched by the method described herein, relative to the reference results. The data generated in FIG. 2 demonstrates that all but one sample had an increase in fetal fraction due to enrichment, with the average increase around 2-2.5 fold. This enrichment enables higher multiplexing and/or improved performance (sensitivity/specificity), with a reduced failure rate also expected. The enrichment may also enable NIPT at earlier stage in pregnancy when fetal fraction is lower. Without being bound by theory, it is believed that the one sample not enriched may either have not actually been enriched, or simply that the reference result was overestimated and the enriched fetal fraction % was slightly underestimated, due to chance. Such enrichment will likely have the effect of significantly improving performance for microdeletion (Mdel) testing. To date, Positive Predictive Values (PPV) are relatively poor in NIPT for Mdels. Enriching fetal fraction could correct for this poor performance and improve PPVs.

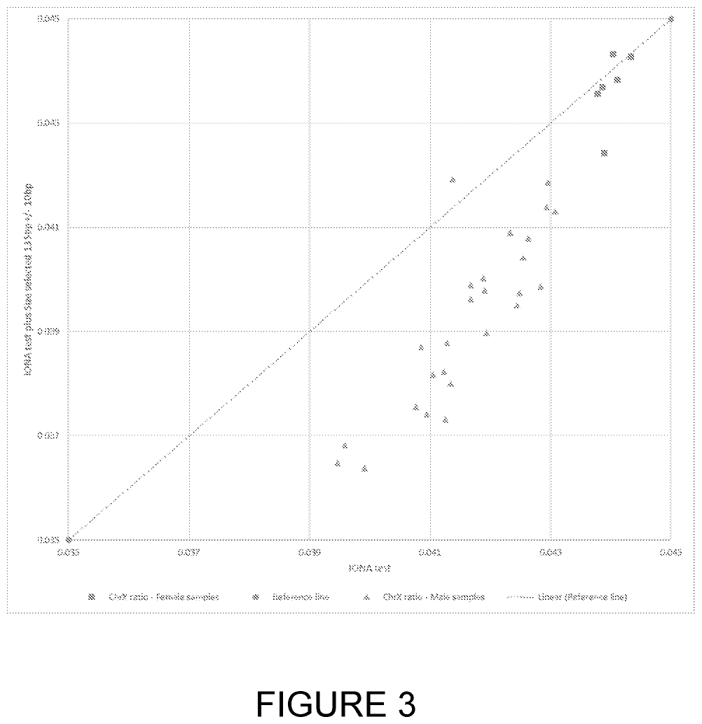

FIG. 3 illustrates the effect of enrichment using the fragment size enrichment method on chromosome X ratios. The Chromosome X ratios for females fetal samples clusters around the expected values; i.e., no change relative to the reference result, whereas the chromosome X ratios are significantly decreased relative to the reference result. The results shown in FIG. 3 for the male samples demonstrate that all but one show decrease in chromosome X ratio using the enrichment method. This is as expected as an increase in the fetal fraction in male fetal samples would lead to a corresponding increase in the Y chromosome ratio and therefore a decrease in the X chromosome ratio of the sample. One sample shows a minor increase (i.e., therefore not enriched) in X chromosome ratio. The results shown in FIG. 3 for the female samples demonstrate that 5 of 6 sit on the reference line at the top right of the figure, i.e., the enrichment method has no effect on the X ratio in female fetal samples (as expected). One sample shows some enrichment. It is possible this one was actually a low fetal fraction male sample in the first place, which was labelled as a female sample in error. The data therefore also demonstrates that the enrichment method could be utilised to improve the accuracy of sex determination testing.

In isolation, the data in FIG. 1, 2 or 3 could have been due to the result of an artefact caused by the size selection of samples to the very narrow desired range. In combination, through increasing Chromosome 21 ratios in T21 samples (with no effect on euploid samples), the decrease in Chromosome X ratio in male samples and the minimal impact on Chromosome X ratios in female samples, along with corresponding increases in fetal fraction estimates, demonstrate the substantial enrichment of fetal DNA in these samples.

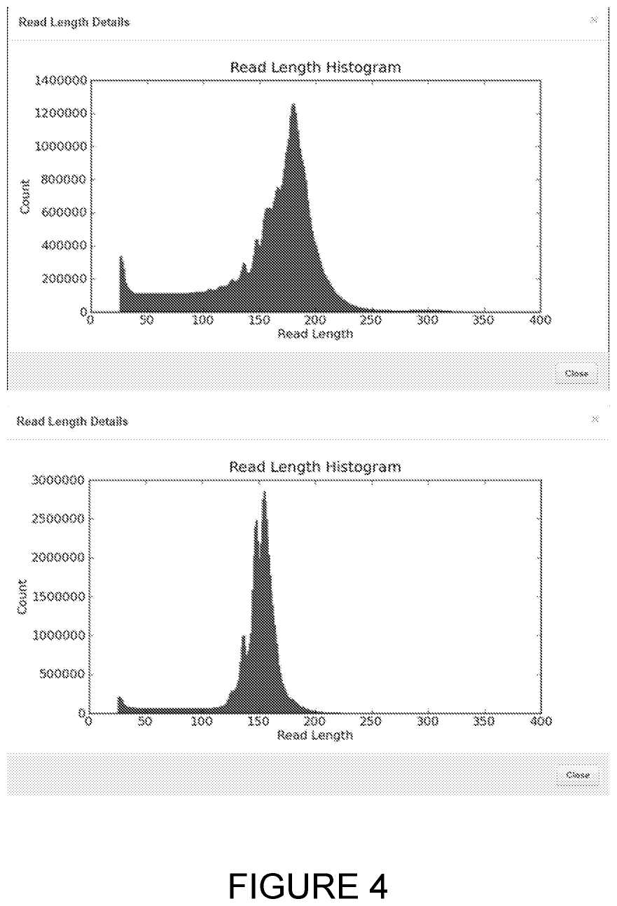

FIG. 4 illustrates the fragment size profiles of a typical whole genome sequencing run (top) and a profile of samples processed using Ranger Technology™. After using the enrichment method, the fragment size distribution of sequenced sample is significantly narrower, with most fragments falling within a 20-30 bp range, centred around a target of 135 bp DNA sample fragment size. Note: fragment sizes also include 13 bp of adaptor sequence.

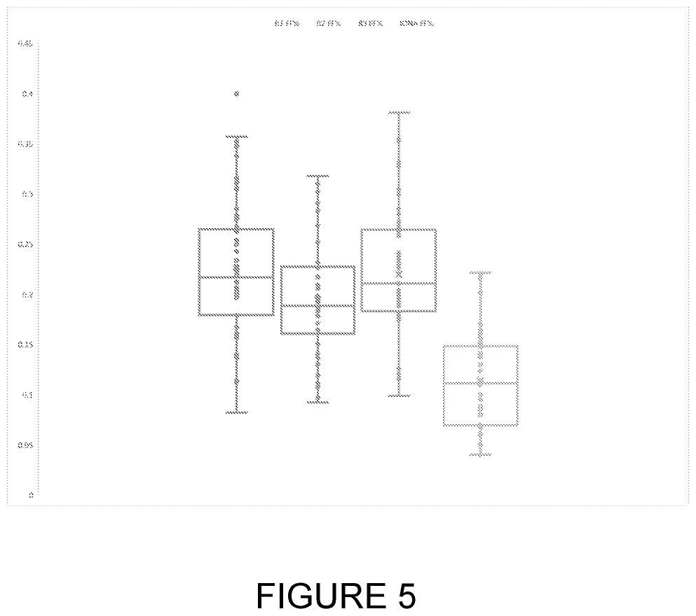

FIG. 5 shows the repeatability of the enrichment method. The same set of samples were processed three times to a target range of 135 bp +/−10 bp using the same methodology (left hand three distributions in the figure). In all three cases, fetal fraction values were comparable across experiments and were higher than the reference control (right hand distribution in the figure).

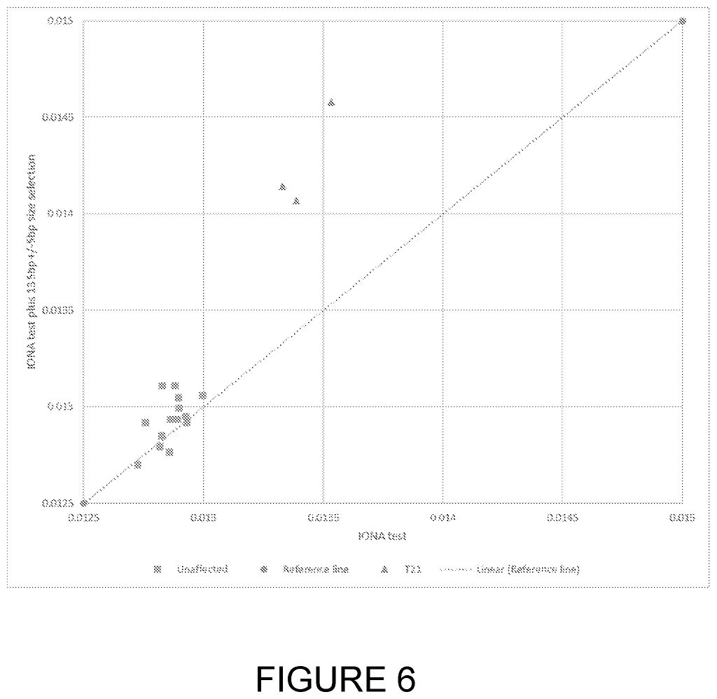

FIG. 6 illustrates the effect of enrichment using the fragment size enrichment method on chromosome 21 ratios using a narrower size selection range around the 135 bp target (+/−5 bp). The ratios for unaffected (euploid) samples clusters around the expected values; i.e., no change relative to the reference result, whereas the chromosome 21 ratios are significantly increased relative to the reference result.

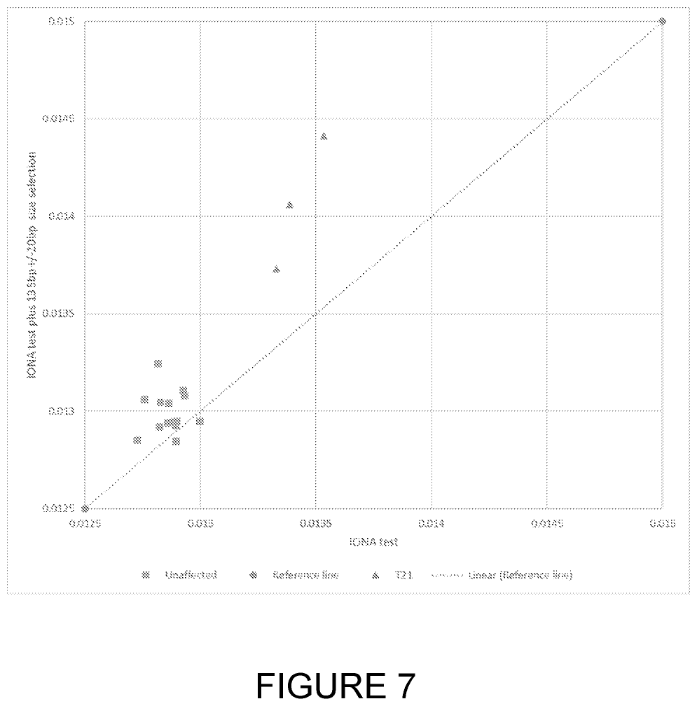

FIG. 7 illustrates the effect of enrichment using the fragment size enrichment method on chromosome 21 ratios using a wider size selection range, of 135 bp +/−20 bp. The ratios for unaffected (euploid) samples clusters around the expected values; i.e., no change relative to the reference result, whereas the chromosome 21 ratios are significantly increased relative to the reference result. However, although the difference in chromosome ratios between unaffected (euploid) and trisomy samples is increased relative to the reference result, the difference appears to be marginally less pronounced than when using the +/−5 or +/−10 bp target capture range, though enrichment is still clearly apparent relative to the reference test method.

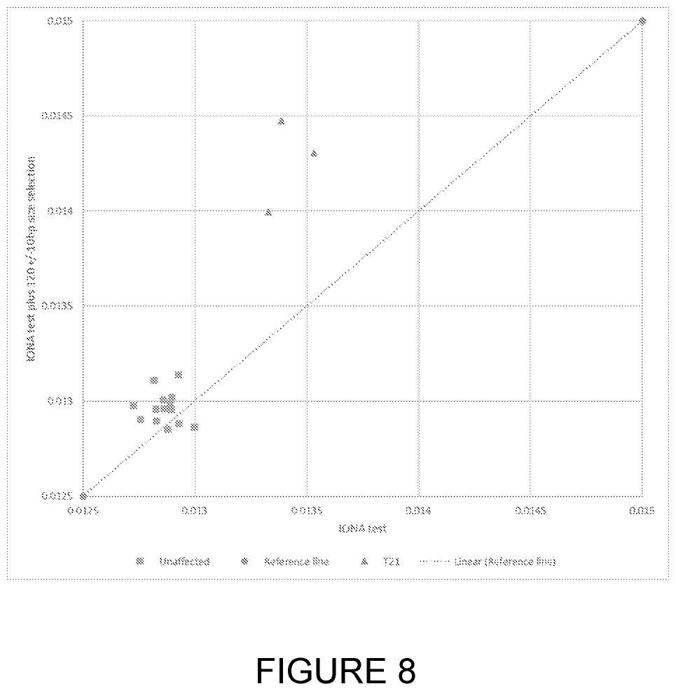

FIG. 8 illustrates the effect of enrichment using the fragment size enrichment method on chromosome 21 ratios using an alternative size selection range (120 bp +/−10 bp). The ratios for unaffected (euploid) samples clusters around the expected values; i.e., no change relative to the reference result, whereas the chromosome 21 ratios are significantly increased relative to the reference result, in a manner that is comparable to the 135 bp +/−10 bp target region. These results demonstrate that targeting fragment sizes as low as 80 bp and as high as 150 bp would be reasonably expected to provide equivalent results.

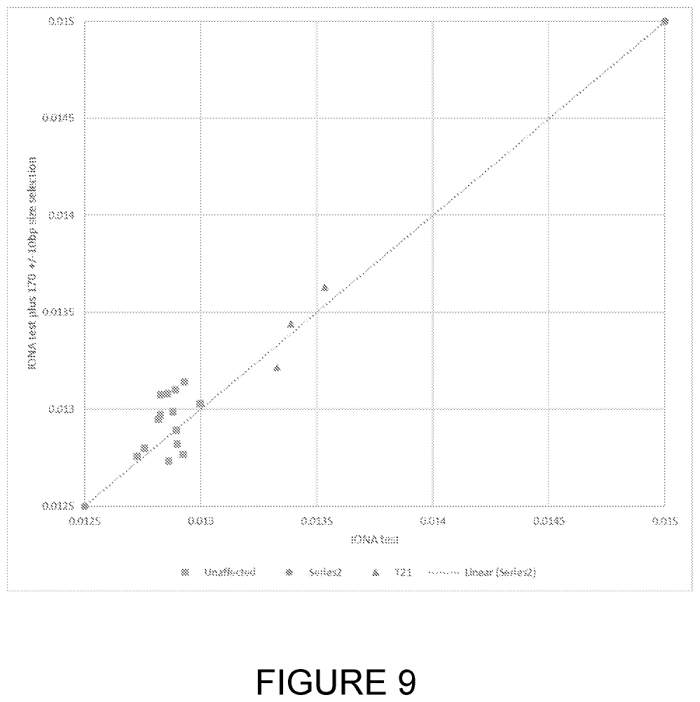

FIG. 9 illustrates the effect of enrichment using the fragment size enrichment method on chromosome 21 ratios using a higher base pair target value (170 bp +/−10 bp). Chromosome 21 ratios for the trisomy samples are relatively unchanged compared with the reference result. However, the euploid samples display more variability in the results, which has the effect of reducing the difference in chromosome ratio between euploid and trisomy samples. Therefore, enriching at this fragment size appears to have a detrimental effect on the ability to distinguish between euploid and trisomy samples.

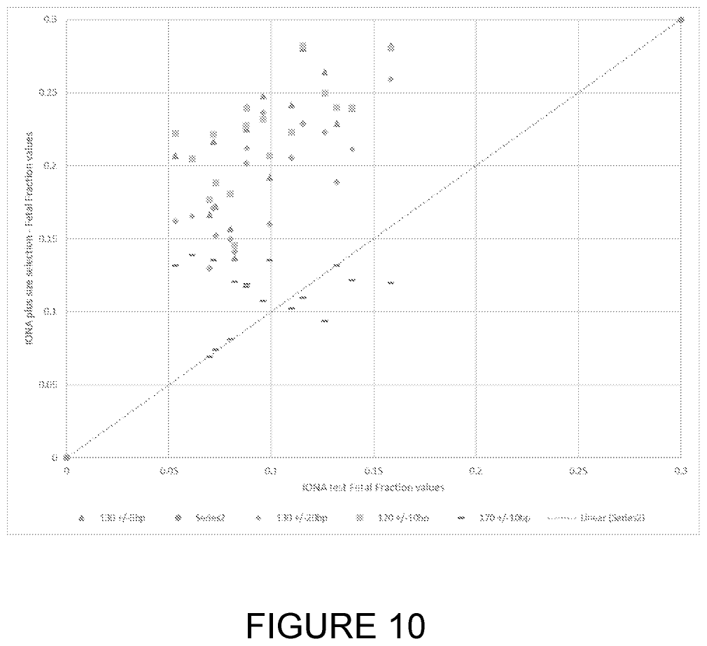

FIG. 10 shows fetal fraction estimates at several fragment size targets and ranges relative to the reference result. The 120 bp and 135 bp targets all display substantial fetal fraction enrichment. The 170 bp target shows a wide variability of effect on fetal fraction, with several samples showing increased fetal fraction and others showing reduced fetal fraction. The data in this figure support the effect on chromosome ratios observed in FIGS. 6-9.

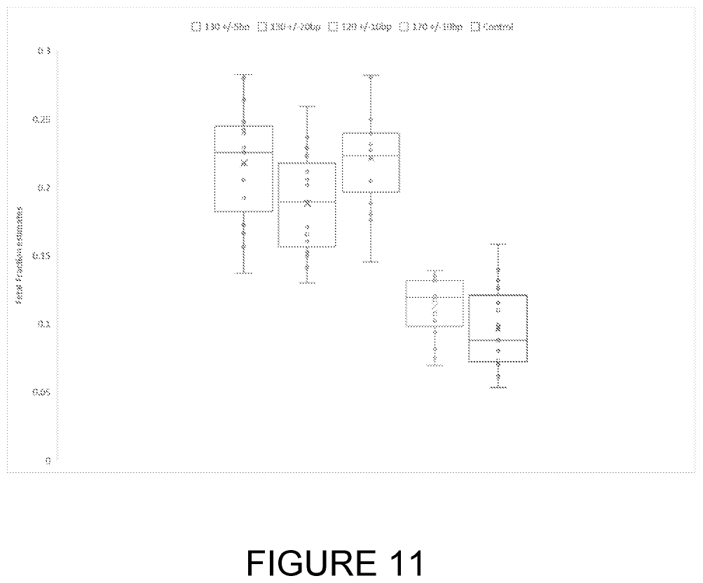

FIG. 11 is a differing manner of displaying the data presented in FIG. 10 which shows fetal fraction estimates at several fragment size targets and ranges in a box and whisker plot. The 120 bp and 135 bp targets all display substantial fetal fraction enrichment relative to the reference result. The 170 bp target shows comparable data to the reference result. The data in this figure support the effect on chromosome ratios observed in FIGS. 6-9.

### 2. Evaluation of Fragment Size Method Embodiment

Samples previously employed in the IONA® clinical performance evaluation study were used to evaluate the method by comparing autosome ratio values for chromosomes 13, 18 and 21 under the updated size-weighted analysis method with those from the baseline unweighted method employed in previous software releases. These samples were drawn from the IONA Study sample collection and other Premaitha Health sample collections.

A total of 405 samples passed IONA® Software validity checks (for sequence data quality and consistency, fragment count density and fetal fraction), and this went forward for use in the comparative analysis. In terms of trisomy status, these samples were distributed as listed in Table 11.

TABLE 11
Trisomy status of samples in the study

| Trisomy status | Sample count |
|---|---|
| Unaffected (euploid) | 351 |
| Trisomy 21-affected (‘T21’) | 40 |
| Trisomy 18-affected (‘T18’) | 9 |
| Trisomy 13-affected (‘T13’) | 5 |

The sequencing data sets corresponding to these samples were extracted from data archives, and analysed using two bioinformatics analysis pipelines:
1. The pipeline as used in validated IONA® Software release 1.6 (with unweighted counting);
2. The updated pipeline as implemented in the updated IONA® Software release 1.7.0 (with fragment size-weighted counting).

The autosome ratios generated in each case for chromosomes 13, 18 and 21 were compared to measure the increase in separation between Unaffected and Trisomy-affected groups, and also assess any change in effective fetal fraction at the point of analysis.

### 2.1 Results

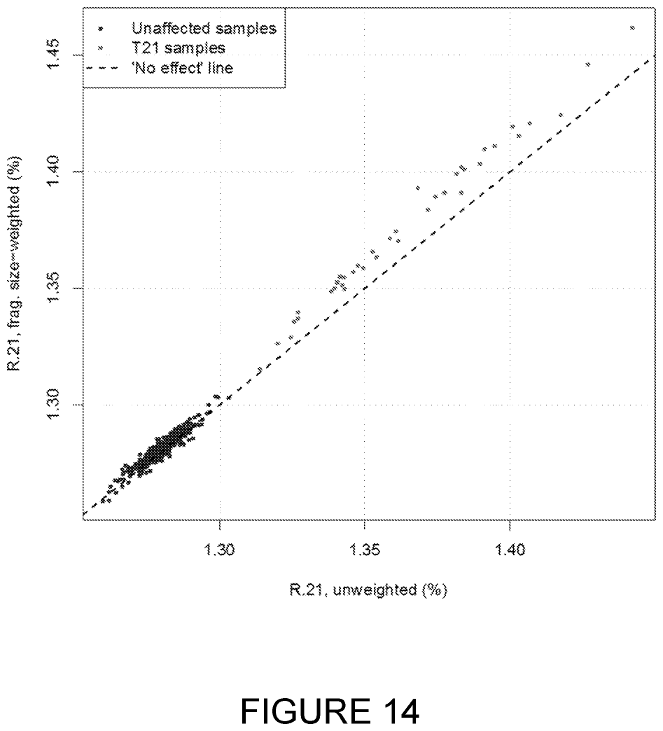

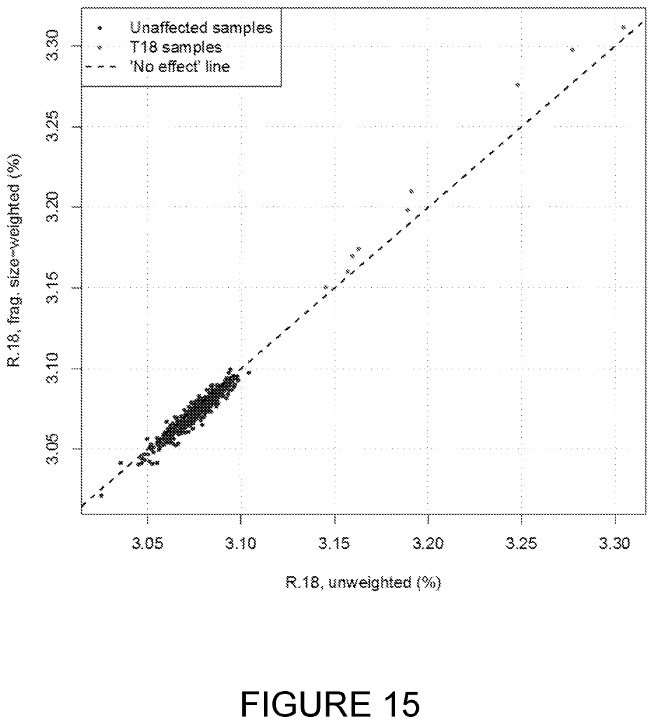

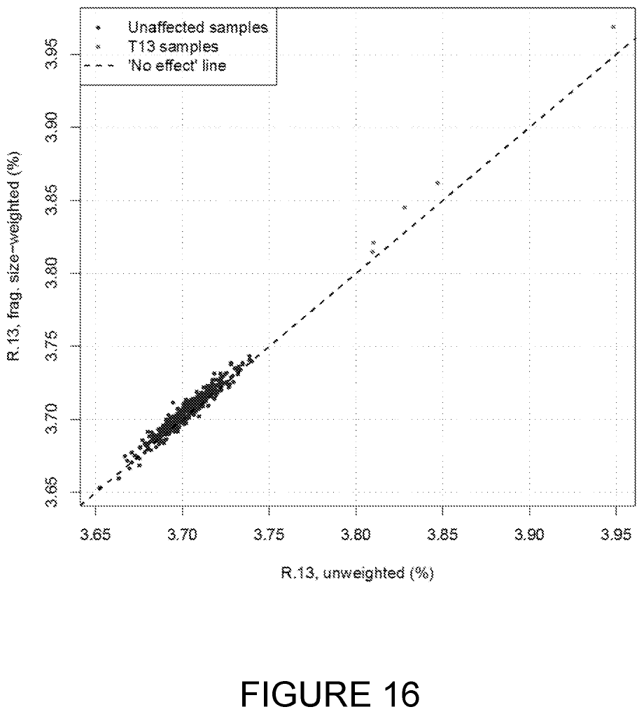

FIGS. 14, 15 and 16 are scatter plots relating the autosome ratios generated by both the unweighted and size-weighted analyses for each sample, for each of the three chromosomes 21, 18 and 13 respectively. These correspond to the tests for trisomies 21, 18 and 13 respectively. Each plot also contains a dotted line through equal autosome ratios between the unweighted and weighted analysis methods.

It can be seen in each case that while the autosome ratios for trisomy-unaffected samples remain clustered around the ‘no effect’ line indicating that the distributions of unaffected samples are unchanged, the autosome ratios calculated for trisomy-affected samples are increased for the size-weighted method compared with the baseline unweighted method.

Additionally, the increase can be seen to be greater for larger autosome ratios than for smaller ones, indicating that the size-weighted method confers a scaling (amplification) effect on trisomy-affected autosome ratios.

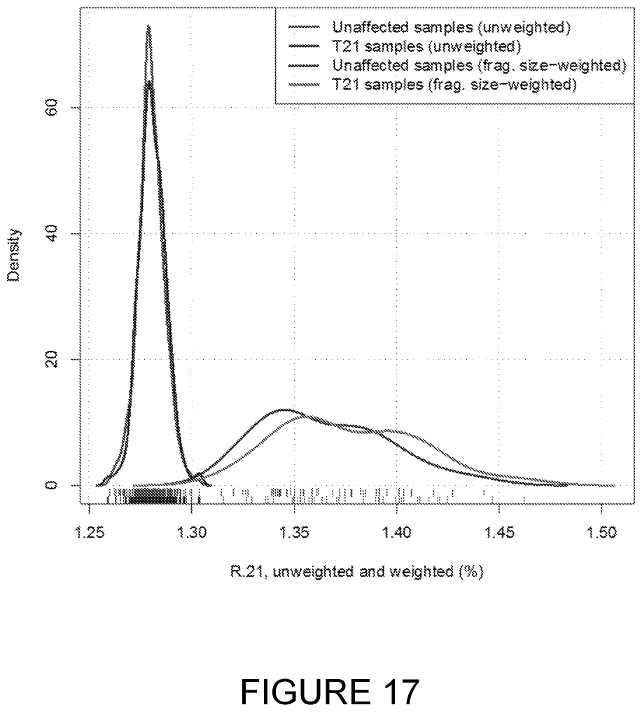

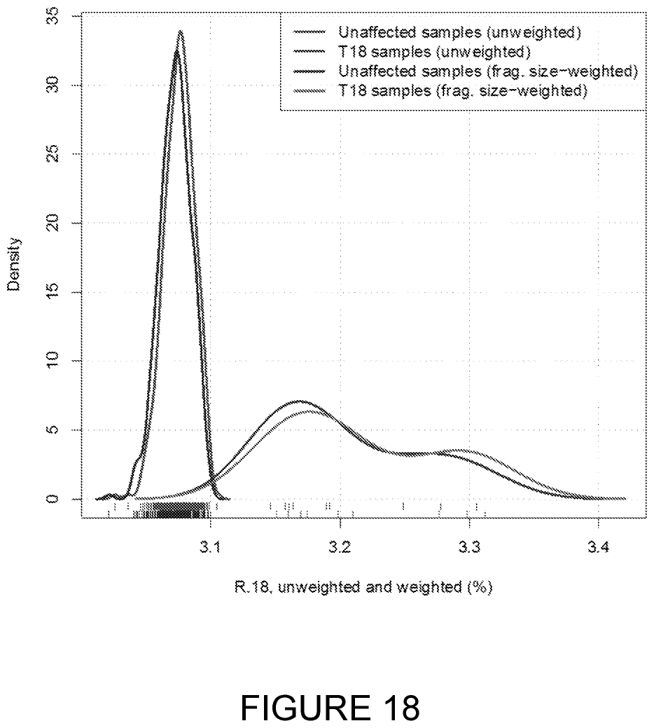

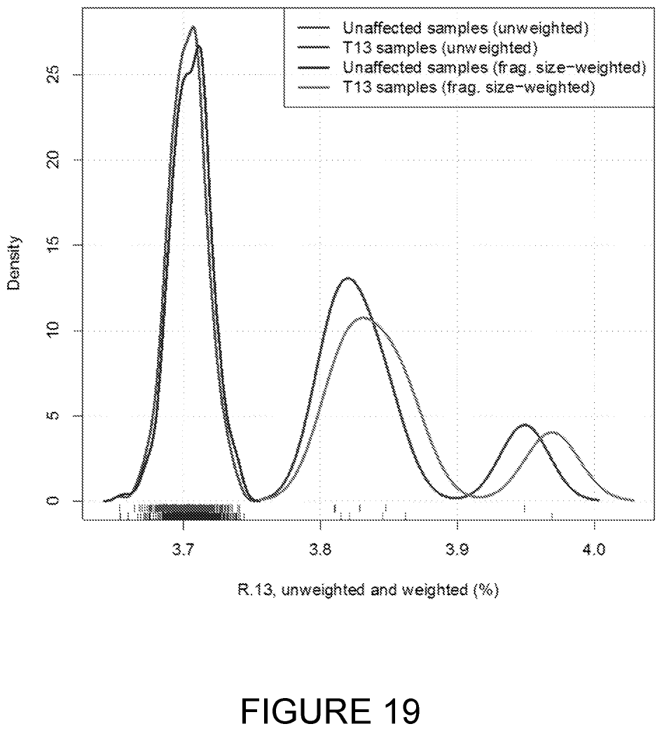

An alternative representation of the data is given by FIGS. 17, 18 and 19; these show empirical distribution functions (kernel density estimates) together with the contributing plotted autosome ratio values, for the trisomy-unaffected and affected groups separately. It can be seen clearly that under the fragment size-weighting method, the affected sample group distributions are shifted and scaled upwards relative to the case of the unweighted method, while unaffected sample groups remain at their original locations.

### 2.1.1 Effect on Performance

A trisomy determination is made in the IONA® Software using a statistical model which has been fitted to the expected unaffected and trisomy group distributions for the population. Sensitivity and specificity performance measures for a system such as the IONA® test are a function of the number of true unaffected and affected cases correctly classified, and the statistical model or cutoff used to determine a result. As such, increasing the separation between unaffected and affected data will have the effect of improving overall performance. Conversely, reducing separation would have a detrimental effect on overall performance. In this study, consistent increased separation was observed between autosome ratio values for the unaffected and affected groups under the fragment size-weighted method when compared with the baseline unweighted method (i.e., between the new and current methods). This increase occurred for trisomy 13, 18 and 21 cases, Therefore, after re-fitting the statistical model used for trisomy determination, an improvement of sensitivity and specificity of the tests for trisomy 13, 18 and 21 is expected in the long term. In particular, this will enable improved detection of trisomy 13, 18 and 21 samples with low fetal fraction, that otherwise could result in a failed test or false negative result, by adjusting the sample result from a likelihood ratio of <1 to a likelihood ratio of >1. Improved sensitivity and specificity with regard to the detection of other abnormalities, such as sex chromosome aneuploidies and microdeletions is also expected.

### 2.1.2 Assessment of Increase in Effective Fetal Fraction

It is possible to calculate the effective fetal fraction seen by the trisomy analysis routines for a trisomy sample, directly from its autosome ratio. Fetal fraction at analysis in a given trisomy sample is proportional to the difference between the autosome ratio for that sample and the expected value (mean) autosome ratio seen for unaffected samples, thus:

$${F}_{eff} = 2 ( \frac{{R}_{{c}_{target}}}{E [ {R}_{{c}_{unaff}} ]} - 1 ) .$$

Here,
c_{target} is the chromosome for the trisomy under test (here c=13, 18 or 21),
R_{c}_{_{target}} is the autosome ratio from analysis for the test trisomy-affected sample,
E[R_{c}_{_{unaff}}] is the expected value (mean) of the autosome ratio for trisomy-unaffected samples, and
F_{eff} is the calculated effective fetal fraction seen by the analysis stage.

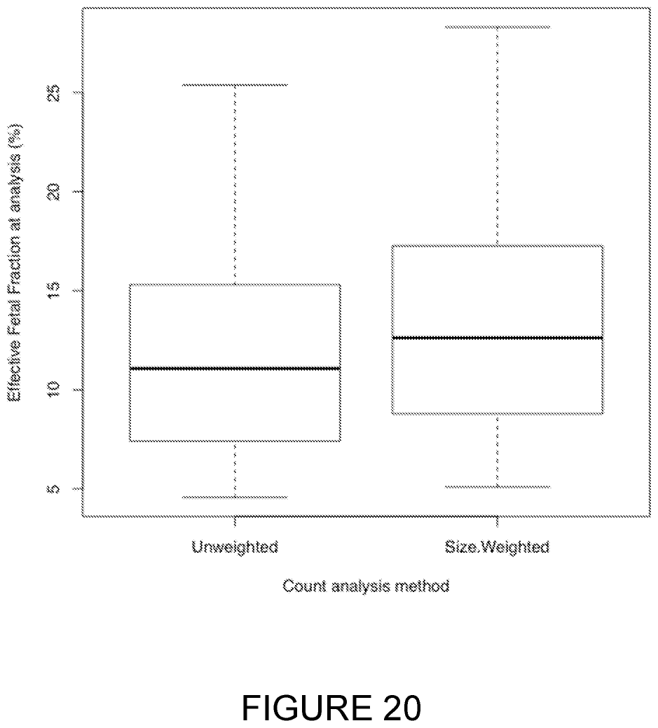

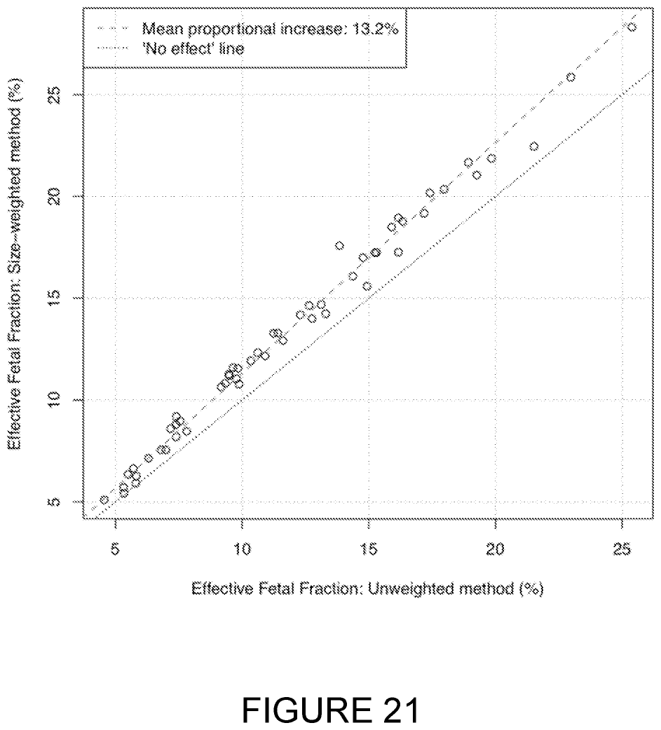

Values of F_{eff} were computed for the 54 trisomy-affected samples in the study data set, where both the existing unweighted and new size-weighted analysis methods had been used to generate autosome ratios. FIG. 20 contains box-and-whisker plots of the distributions of calculated fetal fraction values, as seen at analysis. An increase in median fetal fraction (FF) can be seen (unweighted median FF: 11.1%; size-weighted median FF: 12.6%). Additionally, the distribution of values in the size-weighted case is wider than in the unweighted case, demonstrating the scaling of effective fetal fraction achieved by the improved size-weighted analysis method. FIG. 21 further demonstrates the fetal fraction scaling effect due to the inclusion of size-weighting of each fragment. This plot relates fetal fraction values at analysis for individual trisomy-affected samples as calculated from their autosome ratios, for the original unweighted and new size-weighted analysis cases. The average proportional increase in fetal fraction seen at the trisomy analysis stage due to the improved counting scheme, for all trisomy-affected samples included, is 13.2%.

### 2.2 Conclusions

A method has been developed for incorporation into the IONA® Software to enhance the performance of trisomy determination, or equivalently to reduce the density of sequencing required for a given performance level. The text herein has described a verification exercise conducted to evaluate the updated software routine and confirm that it can meet these requirements.

The study examined the separation between distributions of autosome ratios generated by analysing trisomy-affected and trisomy-unaffected samples using the IONA® test process, using both the existing (unweighted) count analysis method and a new count analysis method which incorporates weighting by fragment size.

Data from 405 clinical samples were considered in the study, consisting of a mix of trisomy-unaffected and trisomy 13, 18 and 21-affected cases. Consistent increased separation was observed between autosome ratio values for the unaffected and affected groups under the fragment size-weighted method when compared with the baseline unweighted method (i.e., between the new and current methods). This increase occurred for trisomy 13, 18 and 21 cases. Therefore, after re-fitting the statistical model used for trisomy determination, for a given sequencing density level, an improvement of sensitivity and specificity of the tests for trisomy 13, 18 and 21 is expected in the long term.

A further investigation has demonstrated that the increase in separation between unaffected and trisomy-affected autosome ratios due to use of the new size-weighting method corresponds to an effective amplification of trisomy sample fetal fraction of 13.2% at the point of trisomy analysis, compared with the existing method.

1. A method of detecting a fetal chromosomal abnormality which comprises the steps of:
(a) isolating nucleic acids from within a biological sample obtained from a pregnant female subject;
(b1) selecting a nucleic acid fragment size value of between 120 bp and 135 bp for optimal fetal fraction;
(b2) isolating nucleic acid fragments having a size within 20 bp of the fragment size value selected in step (b1);
(c) determining a first number of said fragments which align to a target region of a target chromosome and determining a second number of said fragments which align to one or more target regions within reference chromosomes;
(d) calculating a ratio or difference between the first and second numbers;
(e) determining the presence of a fetal abnormality of said target chromosome based on said ratio or difference.

2. The method as defined in claim 1, wherein the fetal chromosomal abnormality is a genetic variation selected from: aneuploidies, duplications, translocations, mutations (e.g. point mutations), substitutions, deletions, single nucleotide polymorphisms (SNPs), chromosome abnormalities, Copy Number Variation (CNV), epigenetic changes and DNA inversions.

3. The method as defined in claim 1, wherein the target chromosome is chromosome 13, chromosome 18, chromosome 21, the X chromosome or the Y chromosome.

4. The method as defined in claim 1, wherein the fetal chromosomal abnormality is a fetal chromosomal aneuploidy, selected from trisomy 13, trisomy 18 or trisomy 21.

5. (canceled)

6. (canceled)

7. The method as defined in any claim 1, wherein the fetal chromosomal abnormality is a chromosomal insertion or a deletion, for example of up to 1 Mb, up to 5 Mb, up to 10 Mb or up to 20 Mb or greater than 20 Mb.

8. The method as defined in claim 1, wherein the target chromosome is a region within a chromosome and the reference chromosome is a region within the same chromosome as the target chromosome.

9. The method as defined in claim 1, which additionally comprises enrichment of the sample for the genomic region suspected to contain the fetal chromosomal abnormality.

10. The method as defined in claim 1, which additionally comprises the steps of:
(i) size-weighting each fragment which aligns to a target region of a target chromosome by calculating the probability (w) of each fragment size (s) being fetal in origin;
(ii) size-weighting each fragment which aligns to one or more target regions within one or more reference chromosomes by calculating the probability (w) of each fragment size (s) being fetal in origin;
(iii) calculating a total target weighted count (Nctarget) by summing the values obtained in step (i);
(iv) calculating a total reference weighted count (Nc) by summing the values obtained in step (ii);
(v) calculating a ratio or difference between the Nctarget and Nc values obtained in steps (iii) and (iv); and
(vi) determining the presence of a fetal abnormality of said target chromosome based on said ratio or difference.

11. A method of predicting the gender of a fetus within a pregnant female subject, the method comprising the steps of:
(a) isolating nucleic acids from within a biological sample obtained from a pregnant female subject;
(b1) selecting a nucleic acid fragment size value of between 120 bp and 135 bp for optimal fetal fraction;
(b2) isolating nucleic acid fragments having a size within 20 bp of the fragment size value selected in step (b1);
(c) determining a first number of said fragments which align to a sex chromosome and determining a second number of said fragments which align to one or more reference chromosomes;
(d) calculating a ratio or difference between the first and second numbers;
(e) determining the gender of said fetus based on whether an excess or equivalence of fragments align to an X chromosome compared to said reference chromosome or whether a Y chromosome is present or absent.

12. The method as defined in claim 11, which additionally comprises the steps:
(i) size-weighting each fragment which aligns to a sex chromosome by calculating the probability (w) of each fragment size (s) being fetal in origin;
(ii) size-weighting each fragment which aligns to one or more reference chromosomes by calculating the probability (w) of each fragment size (s) being fetal in origin;
(iii) calculating a total target weighted count (Nctarget) by summing the values obtained in step (i);
(iv) calculating a total reference weighted count (Nc) by summing the values obtained in step (ii);
(v) calculating a ratio or difference between the Nctarget and Nc values obtained in steps (iii) and (iv); and
(vi) determining the gender of said fetus based on whether an excess or equivalence of fragments align to an X chromosome compared to said one or more reference chromosomes or whether a Y chromosome is present or absent.

13. The method as defined in claim 1, wherein the biological sample is maternal blood, plasma, serum or urine.

14. (canceled)

15. The method as defined in claim 1, wherein the step of isolating in step (a) comprises the preparation of a library of nucleic acid fragments.

16. The method as defined in claim 15, wherein said library preparation comprises the sequential steps of DNA end repair, adaptor ligation, clean up and PCR.

17. The method as defined in claim 1, wherein isolation step (b2) comprises enrichment for nucleic acid fragments having a size within 10 bp of the fragment size value selected in step (b1).

18. The method as defined in claim 1, wherein isolation step (b2) comprises enrichment using size selection selected from gel based size selection or in silico size selection.

19. (canceled)

20. The method as defined in claim 1, wherein step (c) initially comprises sequencing the fragments isolated in step (b2) or subjecting said fragments to digital PCR prior to alignment, wherein said sequencing comprises:
(i) a next generation sequencing system selected from; Life Technologies' Ion Torrent Personal Genome Machine (Ion Torrent PGM) or Ion Proton with a PI or PII Chip, and further derivative devices and components thereof; or Roche 454 (i.e. Roche 454 GS FLX), Applied Biosystems' SOLiD system (i.e. SOLiDv4), Illumina's NextSeq, GAIIx, HiSeq 2000 and MiSeq sequencers, Pacific Biosciences' PacBio RS and Sanger's 3730xl and QIAGENs' GeneReader; or
(ii) a digital PCR system selected from: Quant studio digital PCR system (ThermoFisher) and RainDrop Plus digital PCR system (RainDance technologies).

21. (canceled)

22. (canceled)

23. The method as defined in claim 1, which additionally comprises the step of collapsing duplicate reads from the sequence data obtained prior to alignment step (c).

24. The method as defined in claim 23, wherein step (c) comprises determining a first number of said fragments which uniquely align to a region of a target chromosome and determining a second number of said fragments which uniquely align to one or more target regions within reference chromosomes.

25. The method as defined in claim 1, wherein the alignment step (c) is conducted by IONA®, Bowtie2 or BWA-SW software or software employing Maximal Exact Matching techniques selected from BWA-MEM or CUSHAW2 software.

26. The method as defined in claim 1, which additionally comprises the step of normalizing or adjusting the number of matched hits based on the amount of fetal DNA within the sample.
